SEQUENCE LISTING (18 sequences)

SEQ ID NO: 1 - RNA, 20 nt, synthetic construct
  source 1..20: mol_type = other RNA
cctggggtagagcaggtgag

SEQ ID NO: 2 - RNA, 20 nt, synthetic construct
  source 1..20: mol_type = other RNA
ccactcacctgctctacccc

SEQ ID NO: 3 - RNA, 19 nt, synthetic construct
  source 1..19: mol_type = other RNA
cagggttctggatatctgt

SEQ ID NO: 4 - RNA, 17 nt, synthetic construct
  source 1..17: mol_type = other RNA
ctgcccttacctgggct

SEQ ID NO: 5 - RNA, 20 nt, synthetic construct
  source 1..20: mol_type = other RNA
catcacaggaactttctaaa

SEQ ID NO: 6 - RNA, 20 nt, synthetic construct
  source 1..20: mol_type = other RNA
agctttgaaacaggtaagac

SEQ ID NO: 7 - RNA, 20 nt, synthetic construct
  source 1..20: mol_type = other RNA
ttcgtatctgtaaaaccaag

SEQ ID NO: 8 - RNA, 20 nt, synthetic construct
  source 1..20: mol_type = other RNA
tcaaggcccctcacctcagc

SEQ ID NO: 9 - DNA, 20 bp, synthetic construct
  source 1..20: mol_type = other DNA
ttcacttctccaattgtccc

SEQ ID NO: 10 - DNA, 20 bp, synthetic construct
  source 1..20: mol_type = other DNA
tggctgtggaaagataccta

SEQ ID NO: 11 - DNA, 20 bp, synthetic construct
  source 1..20: mol_type = other DNA
gtcatcctcgaagaagatgg

SEQ ID NO: 12 - DNA, 20 bp, synthetic construct
  source 1..20: mol_type = other DNA
tgtacatcatgaccgacaag

SEQ ID NO: 13 - DNA, 4627 bp, Homo sapiens
  source 1..4627: mol_type = genomic DNA; note = NC_000014.9:22547506-22552132 Homo sapiens chromosome 14, GRCh38.p14 Primary Assembly
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 14 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - DNA, 1448 bp, Homo sapiens
  source 1..1448: mol_type = genomic DNA; note = NC_000007.14:142791694-142793141 Homo sapiens chromosome 7, GRCh38.p14 Primary Assembly
aggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccagccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 16 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - DNA, 1489 bp, Homo sapiens
  source 1..1489: mol_type = genomic DNA; note = NC_000007.14:142801041-142802529 Homo sapiens chromosome 7, GRCh38.p14 Primary Assembly
aggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggtgtgagggaggccagagccacctgtgcacaggtacctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 18 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

(19) United States
(12) Patent Application Publication
(10) Pub. No.: US 2026/0097079 A1
(43) Pub. Date: Apr. 9, 2026

(54) REMOVAL OF ENDOGENOUS TCR CHAINS FOR ENHANCED TCR-BASED IMMUNOTHERAPIES

(71) Applicant: The Trustees of Columbia University in the City of New York, New York, NY (US)

(72) Inventors: Mohsen Khosravi MAHARLOOEI, New York, NY (US); Giorgia ZANETTI, New York, NY (US); Remi CREUSOT, New York, NY (US); Megan SYKES, New York, NY (US)

(21) Appl. No.: 19/415,932

(22) Filed: Dec. 11, 2025

Related U.S. Application Data
(63) Continuation of application No. PCT/US2024/033375, filed on Jun. 11, 2024.
(60) Provisional application No. 63/521,471, filed on Jun. 16, 2023.

(51) Int. Cl. A61K 35/17; A61K 40/11; A61K 40/31; A61K 40/32; A61K 48/00; A61P 37/06; C07K 14/725; C12N 9/22; C12N 15/11; C12N 15/86

(52) U.S. Cl. CPC A61K 35/17; A61K 40/11; A61K 40/31; A61K 40/32; A61K 48/005; A61P 37/06; C07K 14/7051; C12N 9/226; C12N 15/111; C12N 15/86; C12N 2310/20; C12N 2740/15043

(57) ABSTRACT

Methods and compositions for treating a disease in a subject comprising administering an adoptive cell therapy (ACT) to the subject, the ACT comprising delivering a modified lymphocyte comprising a modified T cell receptor (TCR) or a chimeric antigen receptor (CAR);
at least one inactivated endogenous TCRα chain allele; and at least one inactivated endogenous TCRβ chain allele.

### CROSS-REFERENCE TO RELATED APPLICATIONS

This application is a continuation of PCT International Application No. PCT/US2024/033375, filed Jun. 11, 2024, which claims benefit of U.S. Provisional Application No. 63/521,471, filed Jun. 16, 2023, the contents of each of which are hereby incorporated by reference.

### STATEMENT OF GOVERNMENTAL INTEREST

This invention was made with government support under DK123559 and AI045897 awarded by the National Institutes of Health. The government has certain rights in the invention.

### REFERENCE TO SEQUENCE LISTING

This application incorporates-by-reference nucleotide sequences which are present in the file named “240611_92190-A-PCT_Sequence_Listing_AWG.xml”, which is 25,406 bytes in size, and which was created on Jun. 7, 2024 in the IBM-PC machine format, having an operating system compatibility with MS-Windows, which is contained in the XML file filed Dec. 11, 2025 as part of this application.

### BACKGROUND

The disclosures of all publications, patents, patent application publications and books referred to in this application are hereby incorporated by reference in their entirety into the subject application to more fully describe the art to which the subject invention pertains.

TCR-based T cell therapy provides an alternative to chimeric antigen receptor (CAR) T cell therapy, by targeting intracellular antigens through T cell receptors, allowing for a broader range of cancer targets¹⁻⁵. TCR-based adoptive T cell therapy has become one of the most promising approaches of cancer immunotherapy. However, obstacles to widespread T cell therapy adoption include reliance on patient-specific T cells and significant variability among patients^{6, 7}. Especially in individuals with a history of chemotherapies and/or stem cell transplantation, lymphopenic conditions may result in insufficient T cell numbers and impaired T cell functions⁸⁻¹¹. While allogeneic T cells overcome certain limitations of autologous patient-specific T cells, they do not survive well in allogeneic recipients and can induce graft-versus-host disease (GvHD) due to the lack of tolerance to recipient peptide-MHC complexes. The latter challenge can be addressed by genetically retargeting T cells through introduction of transgenic TCRs and simultaneously knocking out the endogenous TCRs to mitigate the risk of GvHD.^{12, 13}.

In contrast to CARs, TCRs have the capability to recognize antigens derived from both extracellular and intracellular sources within the context of human leukocyte antigen (HLA)¹⁴. This broadens the spectrum of available antigens and enhances tumor specificity by targeting neo-antigens or proteins that promote oncogenesis¹⁵. Yet, the presence of endogenous TCR-α and -β chains in overall T cell populations impedes the correct expression of transgenic TCRs, due to mixed TCR dimer formation and competition for binding to the CD3 signaling complex.^{16, 17}. Mispaired TCR variants arising from the heterodimeric structure of transgenic and endogenous receptors lead to reduced surface expression of the intended transgenic TCR and poses a safety risk, as mispaired TCRs may cause off-target toxicities, such as autoimmunity^{18, 19}. Moreover, the TCR forms a complex with CD3 in the endoplasmic reticulum before surface expression²⁰. Consequently, when an endogenous TCR is present, transgenic TCRs compete for association with CD3^{21, 22}.

Several methodologies have been explored to overcome the issue of TCR competition and mispairing, including generation of affinity-enhanced TCRs²³, engineering of mutations to improve the pairing of transgenic TCRs²⁴, and overexpression of CD3 components²¹. However, complete elimination of mispairing and undisturbed expression of the transgenic TCR was only achieved via the full knock out of endogenous TCRs²⁵⁻²⁷. Legut et al. have shown that by targeting TCR-β constant regions, it is possible to prevent the expression of endogenous TCRs²⁵. They targeted the sequences in TCR-β constant regions that differed from corresponding sequences in codon-optimized constructs encoding the transgenic TCR, therefore sparing those TCRs. This method did not target TCR-α chain, and is not applicable for transgenic TCRs whose sequence is not codon-optimized. Roth et al. used a different approach which relies on homologous recombination to insert the new TCR construct within the constant region of TCR-α²⁸. In this method, one of the endogenous TCR chains still remains untouched. Additionally, this approach exhibits relatively lower transduction efficiency compared to lentiviral transduction. Moreover, relying on a non-viral delivery system necessitates a sophisticated design to align the new sequence with the remaining parts of the endogenous TCR-α chain. This renders existing TCR viral vectors unusable, demanding new designs for all current TCR constructs.

### SUMMARY

Disclosed is a novel CRISPR-based method that successfully removes both endogenous TCR-α and -β chains without affecting the transgenic TCRs, whether they are codon-optimized or not. The positive impact of endogenous TCR removal using a clinically relevant transgenic TCR targeting the melanoma antigen recognized by T cells-1 (MART-1)¹ is shown. We show that the simultaneous KO of both endogenous TCR-α and -β chains enhances cellular therapy, ultimately improving performance in a preclinical in vivo model for human metastatic melanoma.

A method of treating a disease in a subject comprising administering an adoptive cell therapy (ACT) to the subject, the ACT comprising delivering a modified lymphocyte comprising
a) a modified T cell receptor (TCR) or a chimeric antigen receptor (CAR);
b) at least one inactivated endogenous TCRα chain allele; and
c) at least one inactivated endogenous TCRβ chain allele;
   thereby treating the disease of the subject.

A method of modifying a cell for adoptive cell therapy, the method comprising:
inactivating an endogenous TCRα chain allele and an endogenous TCRβ chain allele
encoded by the cell, preferably by introducing at least one engineered nuclease to the cell, thereby modifying the cell for adoptive cell therapy.

A method for reducing the likelihood of a graft versus host disease in a subject in need of a TCR or CAR-T therapy for a pathology comprising administering to the subject a modified lymphocyte comprising
a modified T cell receptor (TCR) or a chimeric antigen receptor (CAR);
at least one inactivated endogenous TCRα chain allele; and
at least one inactivated endogenous TCRβ chain allele;

thereby treating the pathology in the subject and reducing the likelihood of a graft versus host disease in the subject.

A mammalian immune cell modified by having a modified T cell receptor (TCR) or a chimeric antigen receptor (CAR); at least one inactivated endogenous TCRa chain allele; and at least one inactivated endogenous TCRb chain allele.

A cell modified according to a method disclosed herein for use in adoptive cell therapy.

A pharmaceutical composition comprising a cell modified according to a method disclosed herein.

A method for enhancing the modification of a cell comprising a therapeutic TCR for an adoptive cell therapy, the method comprising:
inactivating an endogenous TCRα chain allele and an endogenous TCRβ chain allele encoded by the cell, preferably by introducing at least one engineered nuclease to the cell,
thereby enhancing expression and/or formation of a therapeutic TCR modifying the cell for adoptive cell therapy.

A method for enhancing the sensitivity of an adoptive cell therapy TCR to an antigen, comprising modifying a cell expressing the adoptive cell therapy TCR by inactivating an endogenous TCRα chain allele and an endogenous TCRβ chain allele encoded by the cell, preferably by introducing at least one engineered nuclease to the cell, thereby enhancing the sensitivity of the adoptive cell therapy TCR to an antigen relative to an otherwise identical cell expressing the adoptive cell therapy TCR but not modified to inactivate an endogenous TCRα chain allele and an endogenous TCRβ chain allele encoded by the cell.

### BRIEF DESCRIPTION OF THE DRAWINGS

FIGS. 1A-1D: Design of CRISPR strategy for removal of endogenous TCRs while preserving the exogenous ones. (A) Schematic representation of CRISPR strategy for removal of TCR-α and -β chains. The gRNAs target the TCR constant regions at the intron-exon boundaries. (B) Surface expression of TCR in Jurkat cells electroporated with CRISPR-Cas9 with different gRNAs. (C) CRISPR TCR KO strategy spares transgenic TCRs whether or not they are codon optimized. (D) CRISPR KO works only in proliferating PBMCs.

FIGS. 2A-2E: CRISPR removal of endogenous TCRs in primary human T cells enhances exogenous TCR expression and boosts the functionality of MART-1 TCR-transduced T cells. (A) Optimization of timing and order of MART-1 TCR transduction and CRISPR removal of endogenous TCR-α and TCR-β chains. Expression of MART-1 TCR (tetramer staining) and reporter gene (GFP) was measured in different conditions of lentiviral transduction of MART-1 TCR with and without CRISPR-removal of endogenous TCRs: no lentiviral transduction (No V), lentiviral transduction only (V), simultaneous CRISPR-removal of endogenous TCRs and lentiviral transduction (C+V), and CRISPR followed by lentiviral transduction (C then V). (B) The expression of GFP and MART-1 TCR in primary T cells is stable at day 7 and day 16 after transduction. Compiled results from 3 independent experiments are shown as mean±SEM. One-way ANOVA was used for statistical analysis. Statistically significant differences are shown with * signs (* 0.01<p-value<0.05, **0.001<p-value<0.01, ***p-value<0.001). (C) Flow plots representing antigen-specific activation of MART-1 TCR-transduced T cells, in V, C+V and C then V groups, upon encountering MART-1 peptide presented on HLA-A2+ T2 cells (as evident by CD69 surface upregulation). T2 target cells were loaded with different concentrations of MART-1 peptide and co-cultured with transduced T cells for 24 h. MART-1 TCR-transduced T cells without endogenous TCRs showed higher levels of dose dependent T cell activation response to T2 cells loaded with MART-1 peptide, as assessed by CD69 and IFNγ expression (D) and by CD69 and MART-1 tetramer double positivity (E).

FIGS. 3A-3C: MART-1 TCR-transduced T cells without endogenous TCRs show enhanced activation and killing capability upon interaction with MART-1 peptide-expressing autologous LCLs. (A) Overview of the generation of LCL cells engineered to express MART-1 peptide (H3) to use them as autologous stimulators for MART-1 TCR-transduced T cells. (B) In vitro killing assay: MART-1 TCR-transduced T cells co-cultured with autologous LCL cells expressing MART-1 peptide (mCherry+) (n=3 replicates/group). Data are shown as mean±SEM. One-way ANOVA was used for statistical analysis. Statistically significant differences are shown with * signs (* 0.01<p-value<0.05, **0.001<p-value<0.01, ***p-value<0.001). (C) In vitro activation of MART-1 TCR-transduced T cells with autologous LCL cells loaded with different concentrations of MART-1 peptide. (V: virus, C: CRISPR)

FIGS. 4A-4E: Endogenous TCR KO MART-1 TCR transduced T cells exhibit improved specific killing of HLA-A2+ MART-1 peptide (H3+)-expressing K562 tumor cells both in vitro and in vivo. (A) HLA-A2+ T cell proliferation (Ki-67) and activation (HLA-DR and CD25) after 48 h of co-culture with K562 HLA-A2+ H3+, or K562 HLA-A2+ cells as negative control. (B) 48 h cytotoxicity of engineered T cells against a 1:1 mix of K562 HLA-A2+ and K562 HLA-A2+ H3+ (100K total targets). Killing at various effector:target ratios is shown. (C) Overview of strategy for humanized xenograft tumor model. Human immune system NSG MHC I/II KO mice were intradermally implanted with K562 HLA-A2+ H3+ in their right flank and K562 HLA-A2+ in their left flank. T cells were adoptively transferred after 3 days, and the tumor growth was measured for several weeks. (D) Tumor growth curves of K562 HLA-A2+ H3+ and K562 HLA-A2+ over time (n=4-9/group). Data are shown as mean±SEM. Two-way ANOVA was used for statistical analysis. Statistically significant differences are shown with * signs (* 0.01<p-value<0.05). (E) Characterization of infiltrating total and MART-1-specific T cells in the K562 HLA-A2+ H3+ and K562 HLA-A2+ tumoral grafts. Data are shown as mean±SEM. Paired T test was used for statistical analysis. Statistically significant differences are shown with * signs (* 0.01<p-value<0.05). (V: virus, C: CRISPR).

FIGS. 5A-5D: Histological studies show that CRISPR removal of endogenous TCRs increase immune infiltration only in HLA-A2+ MART-1 peptide (H3+)-expressing K562 tumors in HIS mice. Representative histologic images (H&E staining) of K562 HLA-A2+ and K562 HLA-A2+ H3+ tumors. (A) In mice that did not receive any T cells (No V), tumors with or without the expression of MART-1 peptide are similarly devoid of immune cell infiltration. (B) In mice that received transduced T cells (V), there is increased T cell infiltration in K562 HLA-A2+ H3+ tumors compared to the K562 HLA-A2+ control. (C) In C+V and (D) C then V, there is a markedly increased T cell infiltration in K562 HLA-A2+ H3+ tumors compared to the K562 HLA-A2+ control (n=2-3 mice/group).

FIGS. 6A-6G: CRISPR removal of endogenous TCRs from TCR-transduced T cells prevents GvHD after adoptive transfer of these cells to HIS mice. (A) Schematic overview of the experiment to assess GvHD in recipient HIS mice after adoptive transfer of MART-1 TCR-transduced T cells with and without removal of endogenous TCRs. Mice that received C+V and C then V lymphocytes had the (B) lowest GvHD score, (C) least weight loss, and (D) longest survival. (E) Absolute number of CD3+ T cells per μl of peripheral blood of recipient mice at weeks 2 and 4 after adoptive transfer of T cells. C+V and C then V groups had the least T cell expansion. (F) Percentage of MART-1 TCR+ T cells in the peripheral blood of recipient mice at weeks 2 and 4 after adoptive transfer of T cells. (G) Representative histological images (H&E staining) of the lungs and the livers of mice that were adoptively transferred with T cells treated under various conditions. In mice that received No V and V T cells, there is a prominent lymphoid infiltrate which is lacking in mice given C+V or C then V T cells. (n=5-6/group) (V: virus, C: CRISPR). Data in panels B and C were analyzed with the Two-way ANOVA test and shown as mean±SEM. Data in panels E and F were analyzed with the One-way ANOVA test. Log-rank (Mantel-Cox) test was used to analyze the data in panel C survival curve. Statistically significant differences are shown with * signs (* 0.01<p-value<0.05, **0.001<p-value<0.01, ***p-value<0.001, ****p-value<0.0001).

FIGS. 7A-7D: TLA analysis of the integration sites shows widespread integration of the MART-1 TCR transgene. (A) and (C) show genome-wide distributions and characterization of the integration sites of virally introduced MART-1 TCR transgene in human primary T cells of C+V and C then V groups, respectively. In MART-1 TCR-transduced T cells of C+V and C then V groups, integration sites were equally distributed across all chromosomes. No integration sites were found in the TRAC (red circle) or TRBC (green circle) loci. (B) In MART-1 TCR-transduced T cells of C+V group, out of total 179 integration sites, 9 (5%) were found in gene exons, 124 (69%) in gene introns and 2(1%) within 1 kb upstream the genes. (D) In MART-1 TCR-transduced T cells of C then V group, out of total 204 integration sites, 10 (5%) were found in gene exons, 134 (66%) in gene introns and 1 (<1%) within 1 kb upstream the genes.

FIGS. 8A-8B: Comparing different strategies for combined TCR KO with TCR transduction. (A) Both C then V and C+V had increased MART1 TCR transduction efficiency compared to V only. Of note, V then C did not achieve good TCR transduction and was not studied further. (B) Comparable cell counts are achieved between all groups except for V then C.

FIG. 9: Efficiency of MART-1 peptide (H3) transduction in K562 cells.

FIG. 10: Correlation between K562 HLA-A2+ H3+ mCherry+ cells and MART1 tetramer+CD8+ infiltrating T cells using in vivo HIS mice models (see FIG. 4E). As the frequency of CD8+ MART-1+ T cells increase, the number of remaining K562 HLA-A2+ H3+ mCherry+ cells decrease.

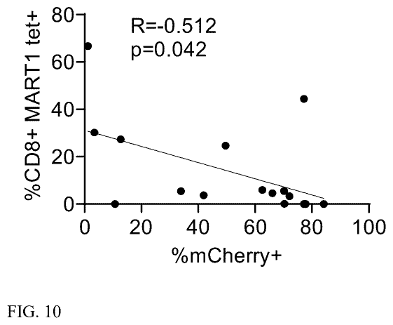

FIG. 11: Quantification of tumor infiltrating lymphocytes in histologic images (H&E staining) of K562 HLA-A2+ and K562 HLA-A2+ H3+ tumors treated under various conditions (see FIG. 5). In mice that did not receive any T cells (No V), tumors with or without the expression of MART-1 peptide are similarly devoid of immune cell infiltration. (b) In mice that received transduced C+V and C then V T cells, there is a markedly increased lymphocyte infiltration in K562 HLA-A2+ H3+ tumors compared to the K562 HLA-A2+ control (n=2-3 mice/group).

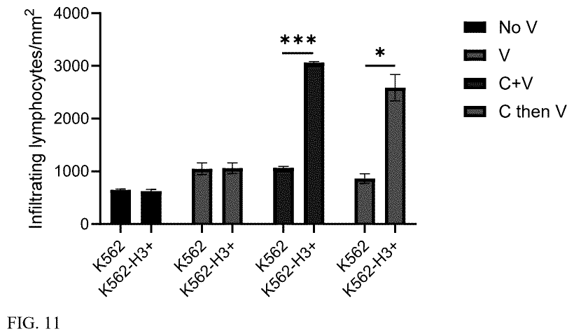

### DETAILED DESCRIPTION

A method of treating a disease in a subject comprising administering an adoptive cell therapy (ACT) to the subject, the ACT comprising delivering a modified lymphocyte comprising
a modified T cell receptor (TCR) or a chimeric antigen receptor (CAR);
at least one inactivated endogenous TCRα chain allele; and
at least one inactivated endogenous TCRβ chain allele;
   thereby treating the disease of the subject.

A method for reducing the likelihood of a graft versus host disease in a subject in need of a TCR or CAR-T therapy for a pathology comprising administering to the subject a modified lymphocyte comprising
a modified T cell receptor (TCR) or a chimeric antigen receptor (CAR);
at least one inactivated endogenous TCRα chain allele; and
at least one inactivated endogenous TCRβ chain allele;
thereby treating the pathology in the subject and reducing the likelihood of a graft versus host disease in the subject.

In embodiments of the methods, the lymphocyte comprises a modified TCR, and the modified TCR comprises a TCRα chain expressed from an exogenous nucleotide coding sequence lacking introns and a TCRβ chain expressed from an exogenous nucleotide coding sequence lacking introns.

In embodiments, each endogenous TCRα chain allele is inactivated in the modified lymphocyte, preferably such that the inactivation knocks out each TCRα chain allele, preferably such that an endogenous, full-length and/or functional TCRα chain is not expressed by the modified lymphocyte.

In embodiments, each endogenous TCRβ chain allele is inactivated in the modified lymphocyte, preferably such that the inactivation knocks out each TCRβ chain allele, preferably such that an endogenous, full-length and/or functional TCRβ chain is not expressed by the modified lymphocyte.

In embodiments, the inactivated endogenous TCRα chain allele is a T Cell Receptor Alpha Constant (TRAC) allele, preferably wherein each TRAC allele is inactivated in the modified lymphocyte.

In embodiments, the inactivated endogenous TCRα chain allele is an inactivated endogenous TRAC allele comprising genetic modification within 50 nucleotides of a TRAC intron-exon boundary.

In embodiments, the TRAC intron-exon boundary is the boundary between (i) TRAC intron 1 and exon 1, (ii) TRAC exon 1 and intron 2, (iii) TRAC intron 2 and exon 2, (iv) TRAC exon 2 and intron 3, (v) TRAC intron 3 and exon 3, and/or (vi) TRAC exon 3 and intron 4, preferably between TRAC intron 1 and exon 1 and/or between TRAC exon 1 and intron 2.

In embodiments, the inactivated endogenous TCRα chain allele comprises a genetic modification to a sequence comprising CAGGGTTCTGGATATCTGT (SEQ ID NO: 3), CTGCCCTTACCTGGGCT (SEQ ID NO: 4), CATCACAGGAACTTTCTAAA (SEQ ID NO: 5), AGCTTTGAAACAGGTAAGAC (SEQ ID NO: 6), TTCGTATCTGTAAAACCAAG (SEQ ID NO: 7), and/or TCAAGGCCCCTCACCTCAGC (SEQ ID NO: 8) located on a TRAC coding or template strand.

In embodiments, the inactivated endogenous TCRβ chain allele is a T Cell Receptor Beta Constant 1 (TRBC1) allele or a T Cell Receptor Beta Constant 2 (TRBC2) allele, preferably wherein each TRBC1 allele and each TRBC2 allele is inactivated in the modified lymphocyte.

In embodiments, the inactivated endogenous TCRβ chain allele is an inactivated endogenous TRBC1 allele and/or an inactivated endogenous TRBC2 allele comprising a genetic modification within 50 nucleotides of a TRBC1 intron-exon boundary and/or a TRBC2 intron-exon boundary.

In embodiments, the TRBC1 intron-exon boundary is the boundary between TRBC1 exon 1 and intron 2, and/or wherein the TRBC2 intron-exon boundary is the boundary between TRBC2 exon 1 and intron 2.

In embodiments, the inactivated endogenous TCRβ chain allele comprises a genetic modification to a sequence comprising CCTGGGGTAGAGCAGGTGAG (SEQ ID NO: 1) and/or CCACTCACCTGCTCTACCCC (SEQ ID NO: 2) located on a TRBC1 coding or template strand and/or a TRBC2 coding or template strand.

In embodiments, the modified T cell receptor is encoded by a codon-optimized nucleotide sequence or a non-codon-optimized nucleotide sequence.

In embodiments, a portion of the inactivated endogenous TCRα chain allele and/or inactivated endogenous TCRβ chain allele is excised, and/or comprises at least two genetic modifications.

In embodiments, the modified TCR is a transgenic TCR and/or an exogenously introduced TCR.

In embodiments, the modified TCR is exogenously introduced into the lymphocyte by transduction of a viral vector, preferably a lentiviral vector, or by delivery of a naked DNA molecule.

In embodiments, the modified TCR is expressed from an exogenous nucleotide sequence integrated into a genomic site at random, in an intron, a safe-harbor site, the inactivated TCRα chain allele, or the inactivated TCRβ chain allele.

In embodiments, the modified lymphocyte comprises a CAR and further comprises at least one inactivated endogenous Class I HLA allele and at least one inactivated endogenous Class II HLA allele, preferably wherein all endogenous Class I and Class II HLA alleles are inactivated, preferably wherein the method further comprises introducing additional agents which inhibit the activity or presence of natural killer (NK) cells.

In embodiments, the lymphocyte is a T cell, a primary T cell, cytotoxic T cell, T helper cell, regulatory T cell, innate-like T cell (iT), natural killer T cell (NKT), γδ T cell, a stem-cell derived T cell, or a iPSc-derived T cell.

In embodiments, the modified lymphocyte is an autologous cell, a lymphocyte isolated from the subject prior to modification of the lymphocyte, or more preferably an allogenic cell, or a lymphocyte isolated from a donor other than the subject prior to modification of the lymphocyte.

In embodiments, the ACT is an autologous ACT or more preferably an allogenic ACT.

In embodiments, the disease is a cancer, autoimmune disease, or an infectious disease. In embodiments, the disease is a cancer. In embodiments, the cancer is a large B-cell lymphoma, a B-cell non-Hodgkin lymphoma, acute lymphoblastic leukemia, mantle cell lymphoma, follicular lymphoma, or a multiple myeloma. In embodiments, the cancer is a solid tumor. In embodiments, the cancer is a melanoma or sarcoma.

In embodiments, the modified lymphocyte is infused into or injected into the subject.

In embodiments, the modified TCR or CAR binds a tumor antigen or a tumor neoantigen.

In embodiments, the modified lymphocyte further comprises an inducible suicide gene.

A method of modifying a cell for adoptive cell therapy, the method comprising:
inactivating an endogenous TCRα chain allele and an endogenous TCRβ chain allele encoded by the cell, preferably by introducing at least one engineered nuclease to the cell,
thereby modifying the cell for adoptive cell therapy.

In embodiments, each endogenous TCRα chain allele is inactivated in the modified cell, preferably such that the inactivating knocks out each TCRα chain allele, preferably such that an endogenous, full-length and/or functional TCRα chain is not expressed by the cell.

In embodiments, each endogenous TCRβ chain allele is inactivated in the modified cell, preferably such that the inactivating knocks out each TCRβ chain allele, preferably such that an endogenous, full-length and/or functional TCRβ chain is not expressed by the cell.

In embodiments, the inactivated endogenous TCRα chain allele is a T Cell Receptor Alpha Constant (TRAC) allele, preferably wherein each TRAC allele is inactivated in the modified cell.

In embodiments, the endogenous TCRα chain allele is inactivated by introducing a genetic modification within 50 nucleotides of a TRAC intron-exon boundary.

In embodiments, the TRAC intron-exon boundary is the boundary between (i) TRAC intron 1 and exon 1, (ii) TRAC exon 1 and intron 2, (iii) TRAC intron 2 and exon 2, (iv) TRAC exon 2 and intron 3, (v) TRAC intron 3 and exon 3, and/or (vi) TRAC exon 3 and intron 4, preferably between TRAC intron 1 and exon 1 and/or between TRAC exon 1 and intron 2.

In embodiments, the inactivated endogenous TCRα chain allele comprises a genetic modification to a sequence comprising CAGGGTTCTGGATATCTGT (SEQ ID NO: 3), CTGCCCTTACCTGGGCT (SEQ ID NO: 4), CATCACAGGAACTTTCTAAA (SEQ ID NO: 5), AGCTTTGAAACAGGTAAGAC (SEQ ID NO: 6), TTCGTATCTGTAAAACCAAG (SEQ ID NO: 7), and/or TCAAGGCCCCTCACCTCAGC (SEQ ID NO: 8) located on a TRAC coding or template strand.

In embodiments, the inactivated endogenous TCRβ chain allele is a T Cell Receptor Beta Constant 1 (TRBC1) allele or a T Cell Receptor Beta Constant 2 (TRBC2) allele, preferably wherein each TRBC1 allele and each TRBC2 allele is inactivated in the modified cell.

In embodiments, the inactivated endogenous TCRβ chain allele is inactivated by introducing a genetic modification within 50 nucleotides of a TRBC1 intron-exon boundary and/or a TRBC2 intron-exon boundary.

In embodiments, the TRBC1 intron-exon boundary is the boundary between TRBC1 exon 1 and intron 2, and/or wherein the TRBC2 intron-exon boundary is the boundary between TRBC2 exon 1 and intron 2.

In embodiments, the inactivated endogenous TCRβ chain allele comprises a genetic modification to a sequence comprising CCTGGGGTAGAGCAGGTGAG (SEQ ID NO: 1) and/or CCACTCACCTGCTCTACCCC (SEQ ID NO: 2) located on a TRBC1 coding or template strand and/or a TRBC2 coding or template strand.

In embodiments, the endogenous TCRα chain allele is inactivated by delivery of a first TCRα-targeting engineered nuclease which binds and affects a first DNA break in the endogenous TCRα chain allele, preferably wherein the first TCRα-targeting engineered nuclease is a CRISPR nuclease complex, a zinc-finger nuclease, or a meganuclease; and delivery of a second TCRα-targeting engineered nuclease which binds and affects a second DNA break in the endogenous TCRα chain allele, preferably wherein the second TCRα-targeting engineered nuclease is a CRISPR nuclease complex, a zinc-finger nuclease, or a meganuclease.

In embodiments, the first TCRα-targeting engineered nuclease is a CRISPR nuclease ribonucleoprotein (RNP) complex comprising a guide RNA molecule which comprises a guide sequence comprising CAGGGTTCTGGATATCTGT (SEQ ID NO: 3), and wherein the second TCRα-targeting engineered nuclease is a CRISPR nuclease ribonucleoprotein (RNP) complex comprising a guide RNA molecule which comprises a guide sequence comprising CTGCCCTTACCTGGGCT (SEQ ID NO: 4).

In embodiments, the endogenous TCRβ chain allele is inactivated by delivery of a first TCRβ-targeting engineered nuclease which binds and affects a first DNA break in the endogenous TCRβ chain allele, preferably wherein the first TCRβ-targeting engineered nuclease is a CRISPR nuclease complex, a zinc-finger nuclease, or a meganuclease; and delivery of a second TCRβ-targeting engineered nuclease which binds and affects a second DNA break in the endogenous TCRβ chain allele, preferably wherein the second TCRβ-targeting engineered nuclease is a CRISPR nuclease complex, a zinc-finger nuclease, or a meganuclease.

In embodiments, the first TCRβ-targeting engineered nuclease is a CRISPR nuclease ribonucleoprotein (RNP) complex comprising a guide RNA molecule which comprises a guide sequence comprising CCTGGGGTAGAGCAGGTGAG (SEQ ID NO: 1), and wherein the second TCRβ-targeting engineered nuclease is a CRISPR nuclease ribonucleoprotein (RNP) complex comprising a guide RNA molecule which comprises a guide sequence comprising CCACTCACCTGCTCTACCCC (SEQ ID NO: 2).

In embodiments, a portion of the inactivated endogenous TCRα chain allele and/or inactivated endogenous TCRβ chain allele is excised, and/or comprises at least two genetic modifications.

In embodiments, method further comprising modifying the cell to express a modified T cell receptor (TCR) or a chimeric antigen receptor (CAR).

In embodiments, the cell is modified to express a modified T cell receptor (TCR), and wherein the modified TCR is a transgenic TCR and/or an exogenously introduced TCR.

In embodiments, the cell is modified to express a modified T cell receptor (TCR), and wherein the modified TCR is exogenously introduced to the cell by transduction of a viral vector, preferably a lentiviral vector, or by delivery of a naked DNA molecule.

In embodiments, the cell is modified to express a modified T cell receptor (TCR), and wherein the modified TCR is exogenously introduced to the cell by transduction of a viral vector at substantially the same time as the at least one engineered nuclease, preferably wherein simultaneous electroporation of the viral vector and the at least one engineered nuclease is performed.

In embodiments, the cell is modified to express a modified T cell receptor (TCR), and wherein the modified TCR is expressed from an exogenous nucleotide sequence integrated into a genomic site at random, in an intron, a safe-harbor site, the inactivated TCRα chain allele, or the inactivated TCRβ chain allele.

In embodiments, the cell is modified to express a modified T cell receptor (TCR), and wherein the modified T cell receptor is encoded by a codon-optimized nucleotide sequence or a non-codon-optimized nucleotide sequence.

In embodiments, the cell is modified to express a modified T cell receptor (TCR), and the modified TCR comprises a TCRα chain expressed from an exogenous nucleotide coding sequence lacking introns, and a TCRβ chain expressed from an exogenous nucleotide coding sequence lacking introns.

In embodiments, the modified cell comprises a CAR and further comprises at least one inactivated endogenous Class I HLA allele and at least one inactivated endogenous Class II HLA allele, preferably wherein all endogenous Class I and Class II HLA alleles are inactivated.

In embodiments, the methods further comprise isolating the cell from a subject or a donor.

In embodiments, the cell is isolated from peripheral blood mononuclear cells (PBMCs), preferably stimulated PMBCs, optionally wherein the stimulation is performed by exposing the PMBCs to any one of CD3, CD28, and/or CD2 antibodies, preferably wherein the stimulation occurs for about a 48-hour period prior to inactivating an endogenous TCRα chain allele and an endogenous TCRβ chain allele encoded by the cell.

In embodiments, the cell is a lymphocyte, T cell, primary T cell, cytotoxic T cell, T helper cell, regulatory T cell, innate-like T cell (iT), natural killer T cell (NKT), 76 T cell, a stem-cell derived T cell, or a iPSc-derived T cell.

In embodiments, the modified cell is an autologous cell, a cell isolated from a subject who will receive the adoptive cell therapy, more preferably an allogenic cell, or a cell isolated from a donor other than a subject who will receive the adoptive cell therapy.

In embodiments, the adoptive cell therapy is an autologous adoptive cell therapy or more preferably an allogenic adoptive cell therapy.

In embodiments, the modified TCR or CAR binds a tumor antigen or a tumor neoantigen.

In embodiments, the modified cell further comprises an inducible suicide gene.

A mammalian immune cell modified by having a modified T cell receptor (TCR) or a chimeric antigen receptor (CAR); at least one inactivated endogenous TCRα chain allele; and at least one inactivated endogenous TCRβ chain allele. In embodiments, the cell has been modified by a method described herein.

A cell modified according to a method disclosed herein for use in adoptive cell therapy. In embodiments, the cell modified is a modified lymphocyte.

In embodiments, the cell modified is a modified T cell, primary T cell, cytotoxic T cell, T helper cell, regulatory T cell, innate-like T cell (iT), natural killer T cell (NKT), γδ T cell, a stem-cell derived T cell, or iPSc-derived T cell.

A pharmaceutical composition comprising a cell modified according to a method disclosed herein. In embodiments, the pharmaceutical composition comprises a pharmaceutically acceptable carrier. In embodiments, the pharmaceutical composition comprises a buffered saline. In embodiments, the pharmaceutical composition is cryopreserved.

A method for enhancing the modification of a cell comprising a therapeutic TCR for an adoptive cell therapy, the method comprising:
inactivating an endogenous TCRα chain allele and an endogenous TCRβ chain allele encoded by the cell, preferably by introducing at least one engineered nuclease to the cell,
thereby enhancing expression of a therapeutic TCR modifying the cell for adoptive cell therapy.

A method for enhancing the sensitivity of an adoptive cell therapy TCR to an antigen, comprising modifying a cell expressing the adoptive cell therapy TCR by inactivating an endogenous TCRα chain allele and an endogenous TCRβ chain allele encoded by the cell, preferably by introducing at least one engineered nuclease to the cell, thereby enhancing the sensitivity of the adoptive cell therapy TCR to an antigen relative to an otherwise identical cell expressing the adoptive cell therapy TCR but not modified to inactivate an endogenous TCRα chain allele and an endogenous TCRβ chain allele encoded by the cell.

In embodiments of the disclosures herein, the cell is a TCR-expressing cell. In embodiments, the cell is a mammalian immune system cell.

Exemplary embodiments of a TCRα chain amino acid sequence, e.g., a T Cell Receptor Alpha Constant (TRAC) amino acid sequence, are provided by UniProtKB/Swiss-Prot Accession No. P01848.2 and SEQ ID NO: 14, as well as related isoforms and variants thereof. Exemplary embodiments of nucleic acid sequences which encode a TCRα chain protein include a T cell receptor alpha constant (TRAC) gene (e.g., NCBI Gene ID: 28755) or the coding sequence within SEQ ID NO: 13 (NC_000014.9:22547506-22552132 Homo sapiens chromosome 14, GRCh38.p14 Primary Assembly), as well as related isoforms or variants thereof.

In some embodiments, a TRAC allele is inactivated by generating a genetic modification within 50 nucleotides of a TRAC intron-exon boundary. An intron-exon boundary as used herein refers to a boundary between an upstream intron and a downstream exon or between an upstream exon and a downstream intron.

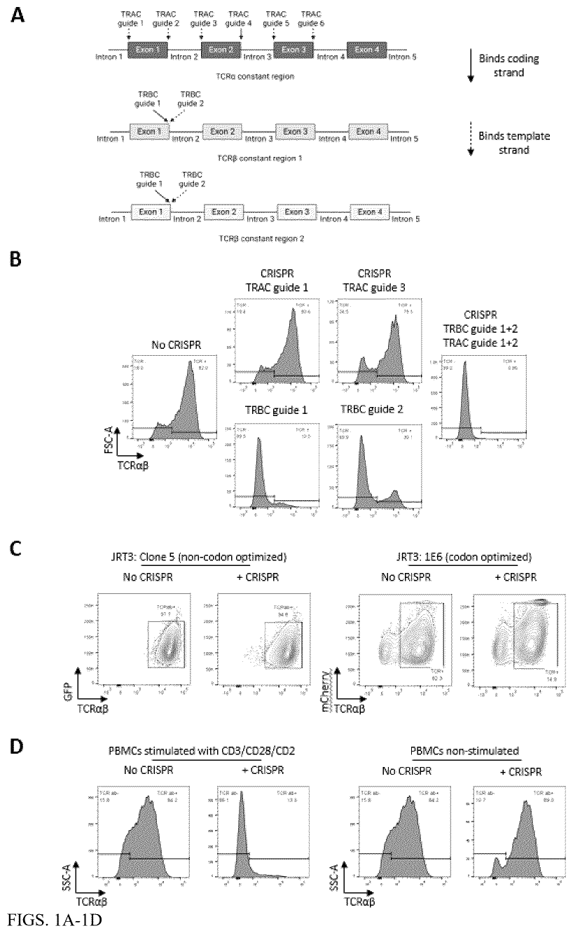

Furthermore, intron 1 of TRAC as used herein is considered to be upstream of the first TRAC exon (i.e., exon 1), as depicted in FIG. 1A. In an exemplary embodiment, intron 1 of TRAC ends immediately upstream of the start of NC_000014.9:22547506-22552132 Homo sapiens chromosome 14, GRCh38.p14 Primary Assembly or the start of TRAC exon 1.

Exemplary embodiments of a TCR chain amino acid sequence, e.g., a T Cell Receptor Beta Constant 1 (TRBC1) amino acid sequence, are provided by UniProtKB/Swiss-Prot Accession No. P01850.4, NCBI GenPept Accession No. AAA60661.1, and SEQ ID NO: 16, as well as related isoforms and variants thereof. Exemplary embodiments of nucleic acid sequences which encode a TCRβ chain protein include a T Cell Receptor Beta Constant 1 (TRBC1) gene (e.g., NCBI Gene ID: 28639) or the coding sequence within SEQ ID NO: 15 (NC_000007.14:142791694-142793141 Homo sapiens chromosome 7, GRCh38.p14 Primary Assembly), as well as related isoforms or variants thereof.

In some embodiments, a TRBC1 allele is inactivated by generating a genetic modification within 50 nucleotides of a TRBC1 intron-exon boundary. An intron-exon boundary as used herein refers to a boundary between an upstream intron and a downstream exon or between an upstream exon and a downstream intron.

Furthermore, intron 1 of TRBC1 as used herein is considered to be upstream of the first TRBC1 exon (i.e., exon 1), as depicted in FIG. 1A. In an exemplary embodiment, intron 1 of TRBC1 ends immediately upstream of the start of NC_000007.14:142791694-142793141 Homo sapiens chromosome 7, GRCh38.p14 Primary Assembly or the start of TRBC1 exon 1.

Additional exemplary embodiments of a TCRβ chain amino acid sequence, e.g., a T Cell Receptor Beta Constant 2 (TRBC2) amino acid sequence, are provided by UniProtKB/Swiss-Prot Accession No. A0A5B9.2, NCBI GenPept Accession No. AAA60662.1, and SEQ ID NO: 18, as well as related isoforms and variants thereof. Exemplary embodiments of nucleic acid sequences which encode a TCRβ chain protein include a T Cell Receptor Beta Constant 2 (TRBC2) gene (e.g., NCBI Gene ID: 28638) or the coding sequence within SEQ ID NO: 17 (NC_000007.14:142801041-142802529 Homo sapiens chromosome 7, GRCh38.p14 Primary Assembly), as well as related isoforms or variants thereof.

In some embodiments, a TRBC2 allele is inactivated by generating a genetic modification within 50 nucleotides of a TRBC2 intron-exon boundary. An intron-exon boundary as used herein refers to a boundary between an upstream intron and a downstream exon or between an upstream exon and a downstream intron.

Furthermore, intron 1 of TRBC2 as used herein is considered to be upstream of the first TRBC2 exon (i.e., exon 1), as depicted in FIG. 1A. In an exemplary embodiment, intron 1 of TRBC2 ends immediately upstream of the start of NC_000007.14:142801041-142802529 Homo sapiens chromosome 7, GRCh38.p14 Primary Assembly or the start of TRBC2 exon 1.

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

Definitions: The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means a mammal. Mammals include canines, felines, rodents, bovine, equines, porcines, ovines, and primates including humans. Thus, the invention can be used in human medicine or also in veterinary medicine, e.g., to treat companion animals, farm animals, laboratory animals in zoological parks, and animals in the wild. The invention is particularly desirable for human medical applications. In a preferred embodiment the subject is a human.

The terms “treat”, “treatment” of a disease or condition, and the like refer to slowing down, relieving, ameliorating or alleviating at least one of the symptoms of the disease.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Results

Design and validation of CRISPR strategy for exclusive removal of endogenous TCRs. To achieve the knockout (KO) of the endogenous TCR without affecting our transgenic TCRs, whether they are codon-optimized or not, we designed guide RNAs (gRNAs) to target the intron-exon boundaries in the constant regions of both TCR-α and -β chains. Since the transgenic TCR constructs do not contain introns, our gRNAs specifically target the endogenous TCR chains. Several gRNAs that targeted the TCR-α constant region (TRAC), and the TCR-β constant regions 1 and 2 (TRBC 1 and 2) were screened (FIG. 1A). Our design of gRNAs targeting TRBC focused on sequences shared between TRBC1 and TRBC2, with the goal of knocking out TCRs containing either of these constant regions. The Cas9 ribonucleoprotein (RNP) complex containing different combinations of gRNAs was delivered via electroporation into Jurkat cell line. Four gRNAs were selected based on their gene disruption efficiency (2 for the TCR-β and 2 for the TCR-α). The combination of 2 gRNAs for each constant region removed the endogenous TCR-α and -β with a higher efficiency compared with a single gRNA targeting each chain in Jurkat cells. The combination of the 4 guide RNAs targeting both chains led to the highest level of TCR removal (more than %95) (FIG. 1B).

To ensure that the gRNAs do not target transgenic TCRs, we tested our strategy on Jurkat, JRT3-T3.5 (TCRβ−/−) cells that were previously transduced with two different exogenous TCRs, one codon optimized (1E6) and the other not codon optimized (Clone 5). The gRNAs did not affect the expression of the either codon-optimized or the non-optimized transgenic TCR (FIG. 1C). These results demonstrate that our CRISPR approach can be used in combination with existing TCR viral vectors.

Primary T cells in an unstimulated state are at rest, leading to the lack of active transcription for many genes. It has been shown that Cas9 binding is influenced by DNA packaging, with actively transcribed genes being more efficiently edited compared to non-actively transcribed genes, where DNA is packaged in heterochromatin 29. For the editing of primary human T cells, we initiated the process by activating peripheral blood mononuclear cells (PBMCs) using ImmunoCult containing anti CD3/CD28/CD2 antibodies from STEMCELL Technologies for a 48-hour period. Subsequently, electroporation was conducted using ribonucleoprotein (RNP) complexes comprising recombinant Cas9 loaded with a mixture of our four guide RNAs (gRNAs) specific for TRAC and TRBC. Targeting both the α and β chains resulted in a notably high TCR knockout rate in stimulated cells, as evidenced by flow cytometry analysis (FIG. 1D left). Not surprisingly, the activation of T cells before gene manipulation was essential to achieve a high efficiency of CRISPR/Cas9 gene editing with the same set of gRNAs (FIG. 1D right).

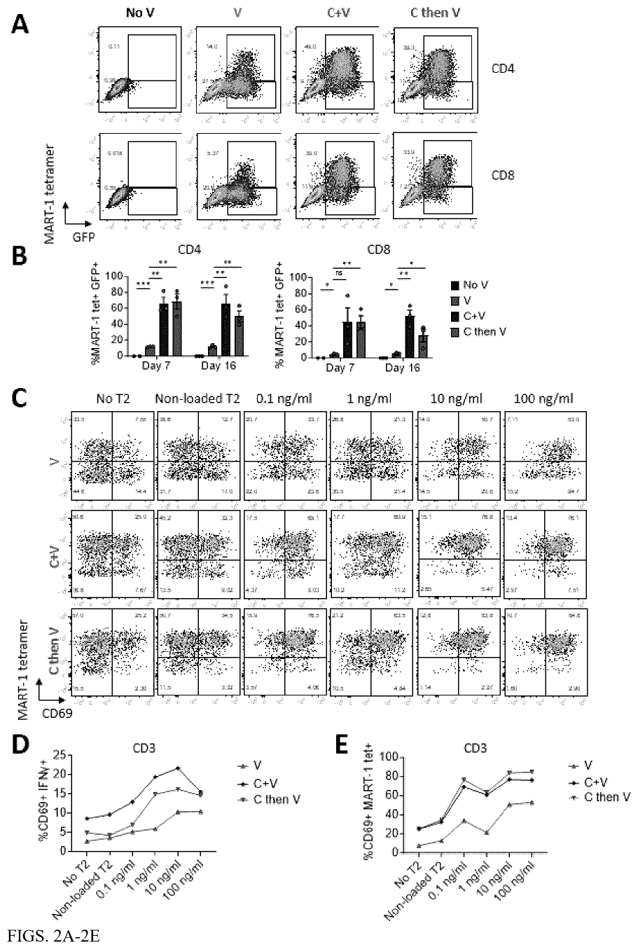

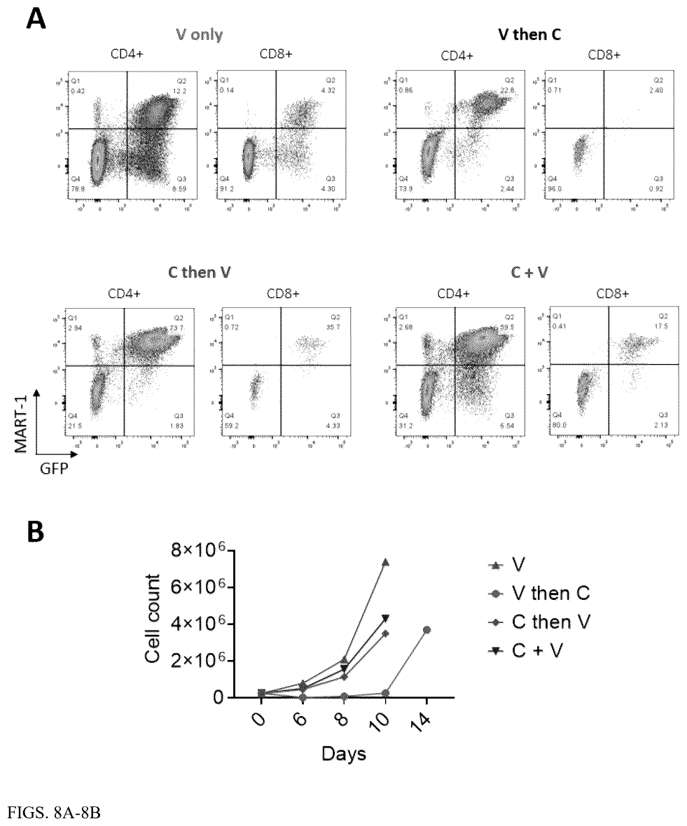

CRISPR removal of endogenous TCRs in primary human T cells enhances exogenous TCR expression. To optimize the timing and sequence of CRISPR-mediated removal of endogenous TCRs and lentiviral delivery of a transgenic TCR, we chose a MART-1 antigen-specific TCR. MART-1 is a clinically significant antigen widely overexpressed in melanomas. 30. A codon-optimized TCR that recognizes the MART-1 antigen in the context of HLA-A2 was employed. We assessed whether introducing the MART-1 transgenic TCR before, simultaneously with, or after CRISPR-mediated removal of endogenous TCRs would enhance transduction efficiency and transgenic TCR expression. While lentiviral transduction followed by CRISPR (with a 1-day difference) did not enhance transduction efficiency compared to treatment without CRISPR-removal of endogenous TCRs (V only), these cells (V then C) exhibited significant cell death and delayed expansion (FIG. 8). Nevertheless, the other two conditions, namely the simultaneous electroporation of the CRISPR RNP complex and TCR lentivirus (C+V group) and the CRISPR-mediated removal of endogenous TCRs followed by lentiviral transduction (C then V), demonstrated markedly enhanced transduction efficiency compared to the V group (FIG. 2A and FIG. 8).

Current methods for transgenic TCR transduction involve coating culture plates with retronectin and centrifuging viral vectors for several hours to bring viral particles close to T cells. In contrast, the C+V approach eliminates the need for this reagent, and the entire process takes place in the same electroporation session, offering a faster, simpler, and more effective alternative. Our approach to removing endogenous TCRs (both C+V and C then V groups) led to a sustained expression of the transgenic TCR over time (from day 7 to 16 of ex vivo expansion) (FIG. 2B). This indicates that TCR-negative T cells (non-transduced T cells that lost their endogenous TCRs) expanded at similar rates to transduced T cells in response to homeostatic cytokines added to the culture (IL-2, IL-7, and IL-15), even in the absence of continuous TCR signaling following the initial TCR activation (FIG. 2B).

In various experiments, the ratio of MART-1 tetramer-positive to tetramer-negative cells within the transduced T cells (GFP+) ranged from 0.5 to 2 in the V group, indicating that a significant proportion of transduced human T cells did not express the intended transgenic TCR. These tetramer-negative GFP+ cells may include T cells with novel specificities, where endogenous and exogenous TCR chains could form pairs. In contrast, in the C+V and C then V conditions, where endogenous TCRs were removed, this ratio ranged from 4 to 10 in various experiments, underscoring the crucial role of endogenous TCR removal in mitigating the potential development of autoreactive TCRs with new specificities.

TCR-transduced T cells without endogenous TCRs show improved activation upon recognizing their target p.MHC. To assess activation in response to target antigens, MART-1 TCR-transduced T cells from the V, C+V, and C then V groups were co-cultured with HLA-A2+ T2 cells as antigen-presenting cells (APCs) loaded with varying concentrations of MART-1 peptide. The human-derived lymphoid cell line T2 lacks TAP gene expression and is incapable of presenting endogenous peptides³¹. After 24 hours of co-culture, the expression of CD69 and IFN-γincreased significantly in a dose-dependent manner across all three TCR transduction conditions. Notably, this increase in activation markers was substantially greater in the C+V and C then V groups, where endogenous T cells were removed, compared to the V group (FIGS. 2C, D and E). These data correlate with the increased frequency of MART-1 tetramer positive cells in the C+V and C then V groups (FIG. 2A).

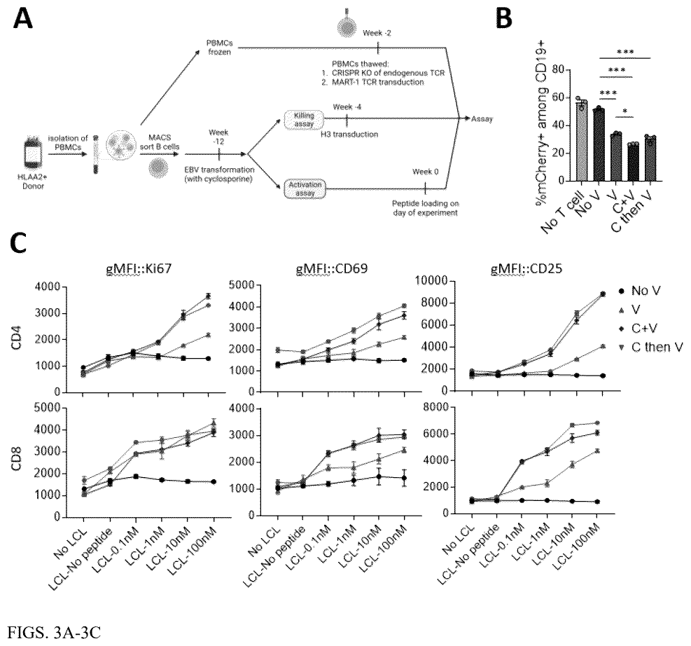

Eliminating endogenous TCRs in TCR-transduced T cells enhances their ability to kill autologous tumor cells expressing their cognate antigens. To establish an autologous model involving T cells and target cells, PBMCs were collected from a healthy HLA-A2+ donor. From this sample, CD19+ B cells were magnetically sorted, and the sorted B cells were then transformed with EBV viral particles to create a B-lymphoblastoid cell line (LCL). The remaining PBMCs from this donor were cryopreserved in multiple aliquots for subsequent in vitro and in vivo studies. A portion of LCL cells were transduced with a lentiviral vector containing the MART-1 peptide (H3-transduced LCLs). Due to low transduction efficiency (ranging from 3-18% in different attempts under varying conditions), H3-transduced LCL cells were sorted using fluorescence-activated cell sorting (FACS) based on the reporter gene (mCherry) present in the H3 vector. These sorted cells were then expanded for downstream assays (FIG. 3A). Despite FACS sorting, the purity of H3-transduced LCL cells (mCherry+) remained approximately 50-60%, and gradually decreased over time.

A vial of frozen PBMCs from the same HLA-A2+ donor was thawed around two weeks before the co-culture experiments, activated with ImmunoCult for 48 hours, and transduced with a lentiviral vector carrying MART-1 TCR in different conditions with and without removal of endogenous TCRs. To measure the impact of removal of endogenous TCRs on killing capacity of TCR-transduced T cells, we co-cultured MART-1 transduced T cells of different groups (V only, C+V and C then V), in addition to non-transduced T cells (No V) with H3-transduced LCL cells, as the target cells. After a 48-hour co-culture, we assessed the proportion of mCherry reporter+ cells among total B cells. Notably, T cells from both the C+V and C then V groups demonstrated the highest levels of antigen-specific killing. Particularly, the C+V group exhibited significantly superior killing activity compared to both the untransduced T cells (No V) and V group counterparts (FIG. 3B).

The autologous MART-1 transduction model was limited by both the suboptimal transduction efficiency of LCLs and the progressive decline in MART-1 peptide transgene expression. As an alternative, we also evaluated our engineered T cells against autologous LCLs loaded with varying concentrations of MART-1 peptide. In line with our earlier observations, we noted enhanced T cell proliferation (Ki-67) and activation (CD69 and CD25) in the C+V and C then V groups, where endogenous TCRs were removed, particularly at higher peptide concentrations (FIG. 3C).

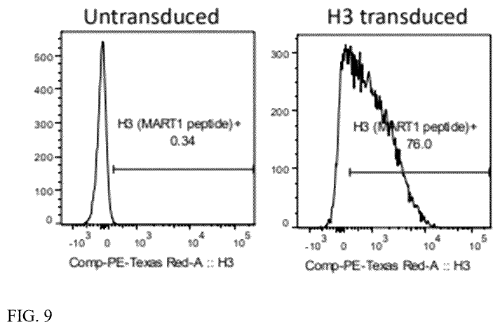

HLA-A2+K562 cells expressing MART-1 peptide proved to assess MART-1 TCR-transduced T cells in vitro. Due to the challenges associated with transduction efficiency and transgene longevity in LCL cells, we explored an alternative tumor model that avoids inducing alloreactions in T cells and has the capacity to present cognate antigens to TCR-transduced T cells. To address this purpose, we utilized the erythroleukemia cell line K562, which lacks HLA class I and II expression on its cell surface³², and therefore does not induce an allogeneic response in donor T cells. To confirm the ability of these cells to present MART-1 peptide to the HLA-A2-restricted MART-1 TCR, we utilized a K562 cell line that had been stably transduced with the HLA-A*0201 gene. Subsequently, we lentivirally engineered these cells to express MART-1 peptide (H3+) (FIG. 9). As T cells from HLA-A2+ donors are tolerant to self HLAs, they are expected to exhibit tolerance to HLA-A2 molecules expressed on K562 cells.

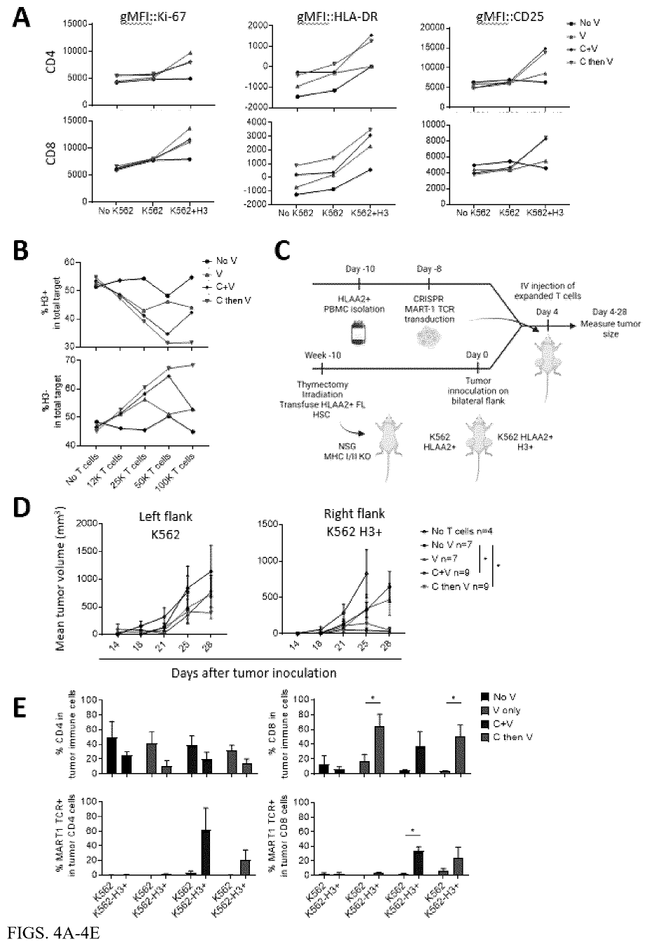

Similar to the prior experiments, we employed lentiviral transduction to introduce the MART-1 TCR into human T cells from an HLA-A2+ donor, with and without the removal of endogenous TCRs (No V, V, C+V, and C then V). Following a 48-hour co-culture, only MART-1 peptide+ (H3+) HLA-A2+ K562 cells induced T cell activation, evidenced by increased Ki-67, HLA-DR, and CD25 expression in MART-1 TCR-transduced T cells. Although T cells in the V group showed some level of activation, the C+V and C then V groups exhibited the highest levels of activation (FIG. 4A), affirming antigen dependence. Lack of activation of No V T cells (no MART-1 TCR) confirmed that endogenous TCRs did not react to the K562 cells. This experiment underscores the superior antigen-specific activation of TCR-transduced T cells lacking endogenous TCRs.

To test the specific killing of MART-1 peptide+ K562 cells by MART-1 TCR-transduced T cells, we employed a 1:1 mixture of HLA-A2+K562 cells with and without transgenic MART-1 peptide (H3+ and H3−), as target cells. Following a 48-hour co-culture, only T cells carrying the transgenic MART-1 TCR were able to eliminate the H3+ cells but spared the H3− controls (FIG. 4B), with the C+V and C then V groups displaying the highest level of killing. Notably, these cells efficiently eliminated H3+ tumor cells in an effector cell dose-dependent manner.

Removal of endogenous TCRs enhances in vivo tumor-killing capability of TCR-transduced T cells. We further analyzed the transgenic T cell functionality in a human immune system (HIS) xenograft tumor mouse model, using NSG-MHC I/II KO mutant mice. This mouse strain combines the features of the nonobese diabetic severe immunodeficient γ chain knockout (NSG) mice with the MHC class I and II molecule deficiency, exhibiting a significant delay in the onset of GvHD after adoptive transfer of human PBMCs³³. These mice underwent thymectomy, received sublethal irradiation (1Gy), and were injected with HLA-A2+ fetal liver CD34+ cells to allow reconstitution with human antigen-presenting cells (APCs) without T cells (FIG. 4C). Approximately 10 weeks later, when the mice were adequately reconstituted with human APCs, we subcutaneously implanted H3+ HLA-A2+ K562 cells in their right flank and H3− HLA-A2+ K562 cells in their left flank. Three days later, the mice received an intravenous injection of either 1 million T cells treated with different conditions (No V, V, C+V and C then V using an HLA-A2+ donor PBMC) or PBS as control (FIG. 4C). Adoptive transfer of MART-1 TCR-transduced T cells in the C+V and C then V groups significantly suppressed the growth of H3+ HLA-A2+ K562 cells compared to transfer of untransduced T cells from the same origin (No V group). Significant tumor suppression was only observed in the groups receiving transduced T cells with endogenous TCR knockout, as the V group showed no suppression of H3+ tumor growth. Thus, removal of endogenous TCR markedly enhanced anti-tumor effects in vivo. Notably, there was no inhibition of the growth of H3− HLA-A2+ K562 tumors, indicating the antigen-specific nature of tumor killing in the H3+ group (FIG. 4D).

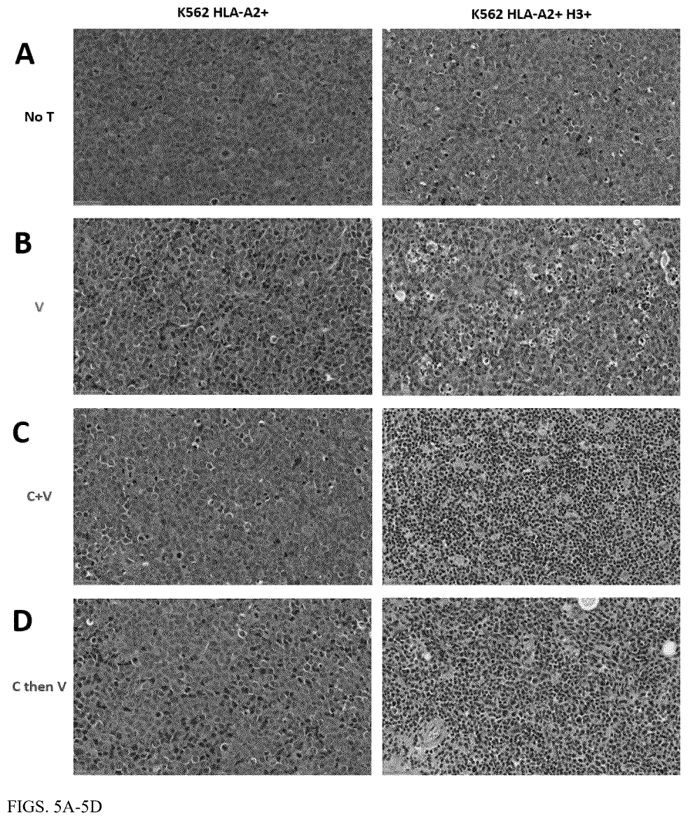

Endpoint tumor examinations revealed a preferential infiltration of CD8+ T cells into H3+ HLA-A2+ K562 masses across all three groups receiving MART-1 TCR-transduced T cells (V, C+V, and C then V). However, a robust infiltration of tetramer+ MART-1 reactive-CD8 cells in H3+ tumors occurred only in the C+V and C then V groups, where the endogenous TCRs were removed (statistically significant in the C+V group) (FIG. 4E). H&E staining of explanted tumors confirmed increased lymphocyte infiltration in H3+ HLA-A2+ K562 tumors of MART-1 TCR-transduced T cell groups, particularly in the C+V and C then V groups (FIG. 5).

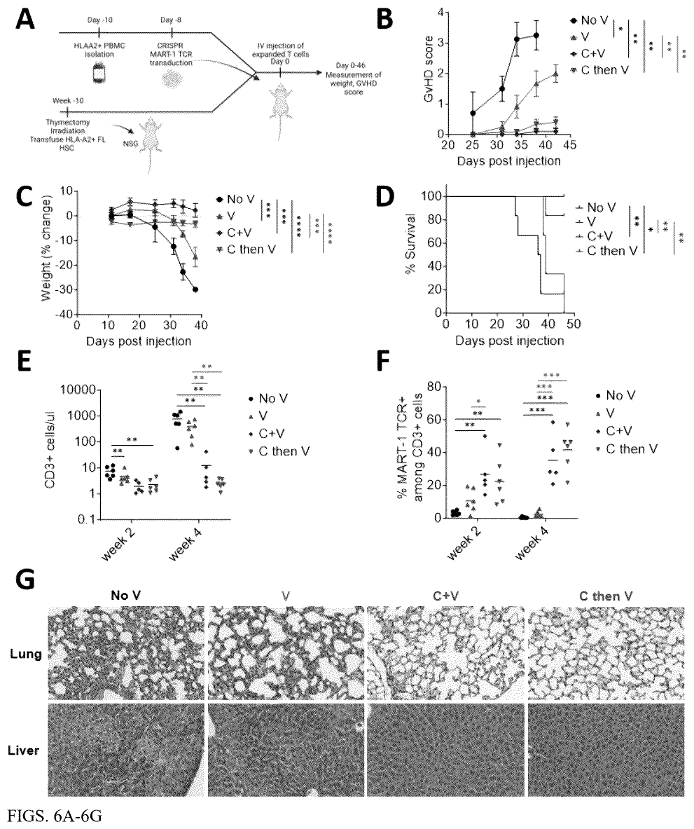

CRISPR removal of endogenous TCR in HIS mice prevents GvHD. To assess whether the elimination of endogenous TCRs mitigated off-target reactivity of TCR-transduced T cells against recipient tissues, we infused NSG mice with C+V, C then V, V, or untransduced T cells. These mice had undergone prior thymectomy and immune reconstitution with fetal liver CD34+ cells, ensuring the presence of human APCs while lacking T cells (FIG. 6A). Among the mice receiving untransduced T cells (No V group), five out of six developed lethal graft-versus-host disease (GvHD) within 46 days of adoptive transfer. All six mice infused with V transduced T cells similarly died due to GvHD. In contrast, none of the mice receiving C+V cells experienced death, and only one recipient receiving the C then V T cells died within the 46-day follow-up, likely unrelated to GvHD (FIG. 6D). Clinical signs of GvHD and significant body weight loss were observed in most mice injected with V or No V T cells, accompanied by characteristic GvHD histopathological findings such as mononuclear cell infiltration in the liver and lung, and bronchial luminal narrowing. No such pathologies were evident in the C+V and C then V groups (FIGS. 6B, C, and G).

Peripheral blood immune cell evaluation at weeks 2 and 4 after adoptive transfer revealed a marked expansion of T cells from week 2 to 4 only in the No V and V groups, in which endogenous TCRs were present (FIG. 6E). This suggests that the reactivity of endogenous TCRs against mouse antigens primarily drives the expansion of these graft-versus-host (GvH)-reactive T cells, while antigen-specific T cells do not significantly expand. Although the proportion of tetramer+ MART-1-reactive T cells in the V group decreased from week 2 to week 4, the proportion of these cells increased over time in the C+V and C then V groups (FIG. 6F), confirming the lack of expansion of (GvH)-reactive T cells.

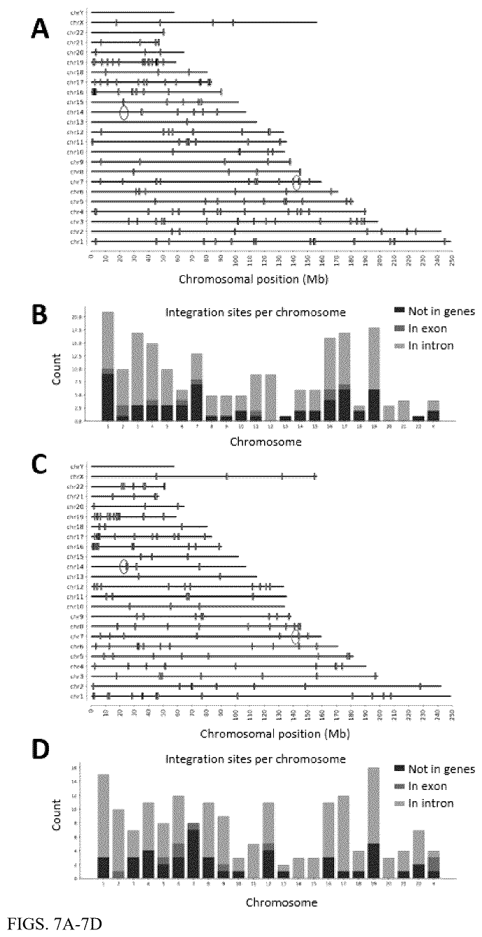

Despite the presence of double-strand DNA breaks with CRISPR, lentivirally transduced transgenic TCRs still exhibit random integration. CRISPR-mediated double-stranded DNA breaks (DSBs) at TRAC and TRBC loci may potentially favor preferential transgene integration into these DSB regions due to DSBs being known to “capture” exogenous DNA^{34, 35}. We hypothesized that this could lead to differences in transgene integration profiles in engineered T cells of C+V versus C then V groups. We used Targeted Locus Amplification (TLA) sequencing to determine the precise location of the MART-1 TCR transgene in the human genome, and the surrounding genomic context. Two primer sets targeting the RRE or GFP regions of the transgene, respectively, were designed for the TLA sequencing (Table 3). Genomic DNA extracted from viable frozen T cells of the C then V and C+V groups were processed for sequencing following the Cergentis protocol³⁶. Reads of genomic DNA fragments were mapped to a reference human genome (GRCh38/hg38) and aligned subsequently. Sequence alignment revealed that no large coverage peaks were seen on the genome for either the analyzed samples, providing no evidence of an integration site that occurs more frequently than other sites. No breakpoints were observed in the TRAC locus (chr14:22,547,506-22,552,156) or in the TRBC gene locus (chr7:142,299,011-142,813,287), ruling out the possibility of preferential transgene insertion at DSBs (FIGS. 7A and 7C). A total of 179 and 204 integration sites were detected in the C then V and C+V samples respectively. Five sequence variants were identified in both samples, indicating they most likely were present in the sequence of the virus before transduction. The integration sites were identified across all chromosomes in intergenic regions, introns and exons, as shown in FIG. 7. Out of a total of 179 integration sites in the C then V sample, 9 (5%) were found in gene exons, 124 (69%) in gene introns and 2 (1%) within 1 kb upstream the genes (FIG. 7B). Out of a total of 204 integration sites in the C+V sample, 10 (5%) were found in gene exons, 134 (66%) in gene introns and 1 (<1%) within 1 kb upstream the genes (FIG. 7D). These findings suggest that CRISPR-induced DSBs at TRAC and TRBC loci do not elevate the likelihood of transgenic TCR integration at these sites. The majority of integration sites are located within introns, potentially having no direct impact on gene expression. However, given that approximately 5% of integrations occur in exon regions, there exists a possibility that certain oncogenes or tumor suppressor genes may be influenced by lentiviral integration, potentially leading to the development of oncogenic T cells expressing transgenic TCRs.

TABLE 1
Sequence of guide RNAs targeting TRAC and TRBC
intron/exon boundaries as indicated in FIG. 1A.

| TCR gene | Guide | Sequence |
|---|---|---|
| TRBC1 + | 1 | CCTGGGGTAGAGCAGGTGAG (SEQ ID NO: 1) |
| TRBC2 | | |
| TRBC1 + | 2 | CCACTCACCTGCTCTACCCC (SEQ ID NO: 2) |
| TRBC2 | | |
| TRAC | 1 | CAGGGTTCTGGATATCTGT (SEQ ID NO: 3) |
| TRAC | 2 | CTGCCCTTACCTGGGCT (SEQ ID NO: 4) |
| TRAC | 3 | CATCACAGGAACTTTCTAAA (SEQ ID NO: 5) |
| TRAC | 4 | AGCTTTGAAACAGGTAAGAC (SEQ ID NO: 6) |
| TRAC | 5 | TTCGTATCTGTAAAACCAAG (SEQ ID NO: 7) |
| TRAC | 6 | TCAAGGCCCCTCACCTCAGC (SEQ ID NO: 8) |

TABLE 2
List of flow cytometry antibodies used in the study.

| Marker | Fluorophore | Clone and Vendor |
|---|---|---|
| CD8 | APC-Cy7 | SK1, BD Pharmingen |
| CD4 | AF700 | OKT4, Tonbo |
| CD3 | BV650 | UCHT1, BD Pharmingen |
| CD45 | AF660 | HI30, BioLegend |
| CD69 | BV650 | FN50, BioLegend |
| CD25 | BV711 | M-A251, BioLegend |
| CD19 | BV510 | HIB19, BioLegend |
| IFN-γ | APC | 4S.B3, BioLegend |
| TCRαβ | PE | IP26, BioLegend |
| Ki-67 | APC | Ki-67, BioLegend |
| HLA-DR | BV605 | G46-6, BD Pharmingen |
| MART-1 tetramer | PE | MBL |

TABLE 3
Sets of primers used to perform TLA.

| Primer | Sequence |
|---|---|
| RRE RV | TTCACTTCTCCAATTGTCCC (SEQ ID NO: 9) |
| RRE Fw | TGGCTGTGGAAAGATACCTA (SEQ ID NO: 10) |
| GFP Rv | GTCATCCTCGAAGAAGATGG (SEQ ID NO: 11) |
| GFP Fw | TGTACATCATGACCGACAAG (SEQ ID NO: 12) |

### DISCUSSION

Adoptive transfer of genetically engineered T cells has emerged as a promising and revolutionary form of cancer immunotherapy. This strategy leverages the power of the immune system to specifically target and destroy tumor cells, offering hope for more effective and personalized cancer treatment^{2, 37}. Genetic retargeting of T cells can be accomplished using two main methods: transduction with a CAR or with a TCR designed for a specific antigen. While CAR-based therapy has demonstrated remarkable success in CD19-positive hematological malignancies³⁸, it is limited to targeting surface-expressed molecules. In contrast, the utilization of specific TCRs enables the targeting of intracellular tumor antigens, a crucial aspect, especially in the treatment of solid tumors³⁹.

Utilizing a transgenic TCR in primary T cells encounters challenges due to the existing endogenous TCRs within these cells. The TCR forms a ternary complex with CD3 for surface expression; hence, in the presence of an endogenous TCR, transgenic TCRs must compete for CD3 association²¹. Additionally, exogenous TCR chains may form heterodimers with their endogenous partner chains. This phenomenon, known as TCR mispairing, not only impedes adequate surface expression of the transgenic TCR but also gives rise to receptors with unpredictable specificities, potentially leading to hazardous autoreactivity¹⁹.

While toxicity due to TCR mispairing has not been observed in clinical trials, murine experiments have reported its potential to induce severe GvHD-like pathology¹⁸. Hence, there is a significant emphasis on devising methods to eliminate endogenous TCRs for the production of TCR-transduced T cells. These approaches, in combination with the engineering of other genes, could also enable the secure utilization of third-party or allogeneic T cell donors to create off-the-shelf T cell products that do not trigger GvHD in recipients, applicable to both CAR and TCR engineering.⁴⁰. Several methodologies have been explored to optimize the expression of therapeutic TCRs and reduce mispairing, such as overexpression of CD3 components²¹, murinization of human TCRs^{41, 42}, inclusion of additional cysteine residues^{43, 44}, codon optimization⁴⁵ and knockdown of the endogenous TCR. Gene editing techniques like Zinc-finger nucleases (ZFNs) and Transcription activator-like effector nucleases (TALENs) initially paved the way for silencing endogenous TCR expression, leading to the creation of antigen-specific T cells^{46 47}. CRISPR is an alternative which unlike its predecessors relies on gRNA sequences rather than protein/DNA attachment.

In this study, our objective was to enhance the functionality of engineered T cells during TCR gene transfer by concurrently knocking out the endogenous TCR while introducing a desired TCR via viral transfer. To achieve this goal, we utilized the CRISPR/Cas9 strategy and designed gRNAs to target the intron-exon boundaries in the constant regions of both TCR-α and -β chains. Our results demonstrated that this approach not only enhances the expression of the therapeutic TCR being inserted, but also significantly improves antigen sensitivity. T cells transduced using this system exhibited superior reactivity to target cells both in vitro and in vivo compared to T cells expressing both endogenous and transgenic TCRs.

To compare the function of TCR-transduced T cells with and without the removal of endogenous TCRs, we had to establish autologous models, lacking alloreactivity of endogenous TCRs against tumor cells. Initially, we used LCLs derived from an HLA-A2+ donor and subsequently incorporated MART-1 peptide via transduction. Autologous T cells from the same donor were then engineered with MART-1 TCR. This approach enabled a direct comparison of the activity between MART-1 TCR-transduced T cells with and without the removal of endogenous TCRs. Although suitable for in vitro functionality assessments, the limited transduction efficiency of LCLs, coupled with the gradual decline in transgenic peptide expression within these cells, constrained their utility for testing the killing capacity of TCR-transduced T cells in vivo.

As an alternative approach, we utilized the K562 tumor cell line, which lacks expression of class I and II HLAs. These HLA-deficient K562 cells were transduced with HLA-A2 and engineered to express MART-1 peptide. Unlike LCLs, K562 cells were notably more amenable to stable transduction with exogenous peptide. We verified that endogenous TCRs from an HLA-A2+ donor did not elicit a reaction against the HLA-A2+ K562 cells, rendering this model suitable for evaluating the efficacy of exogenous TCRs. By implanting HLA-A2+ MART-1 peptide+ K562 cells and administering TCR-transduced T cells into HIS mice containing HLA-A2+ APCs but lacking T cells, we demonstrated that TCR-transduced T cells, in which endogenous TCRs are eliminated, outperform those retaining endogenous TCRs in terms of tumor eradication and infiltration of antigen-specific T cells into the tumor site. The TCR-modified lymphocytes remained in circulation for at least four weeks post-infusion and sustained high levels of expression of the tumor-specific TCR. This indicates the potential for long-term stability of these cells in vivo.

Significantly, in our HIS mouse model, we observed no signs of GvHD following the infusion of TCR-transduced T cells from which endogenous TCRs were removed. Conversely, infusion of TCR-transduced T cells retaining endogenous TCRs led to lethal GvHD in all recipient mice. Moreover, cells edited for both TCR-α and -β displayed notably diminished activity against nonspecific targets compared to unedited T cells, underscoring the importance of preventing the formation of mispaired TCRs and suggesting that such modifications mitigate the risk of autoimmune diseases. The efficient platform described in this study may be extended to off-the-shelf applications involving simultaneous multiple genetic ablations associated with HLA I/II expression, including β-2 microglobulin (B2M) and class II major histocompatibility complex transactivator (CIITA) genes. This approach holds promises for generating universal off-the-shelf TCR-specific T cells with minimal risks of GvHD and T cell graft rejection^{50, 51}.

The broad adoption of gene-editing technology for immunotherapy appears imminent, highlighted by the recent success of off-the-shelf allogeneic CAR19 T cells in inducing remission of B-cell acute lymphoblastic leukemia^{52, 53}. In essence, it is probable that TCR knockout will yield clinically beneficial T cells devoid of the risks associated with TCR mispairing, showcasing enhanced potency and sensitivity compared to current trial products.

While lentiviruses are known to integrate randomly into the genome, the risk of insertional mutagenesis varies among different cell types, particularly hematopoietic stem cells and T cells. Studies have shown that integrated lentiviruses are notably more abundant in transcriptionally active regions^{54, 55.} leading to the perception of lentiviral engineering of T cells as a safe approach, resulting in FDA approval for multiple lentivirus-based CAR T cell products. However, recent research has highlighted potential risks of insertional mutagenesis in T cells⁵⁶⁻⁵⁸. We tested if a double-strand DNA cut induced by CRISPR during lentiviral transduction would promote the insertion of an external TCR gene into the cut sites (endogenous TCR). Nonetheless, although lentiviral integration sites were dispersed across all chromosomes without bias toward the cut sites, we did not observe any preferential integration at the cut sites in the group of transgenic T cells where lentiviral transduction and CRISPR were simultaneously introduced (C+V group), compared to the group where lentiviral transduction followed CRISPR. These findings indicate that the integration mechanisms of lentiviruses remain unaffected by the double-strand DNA cut introduced by CRISPR.

### Methods

### Lentiviral Vector Production

A second-generation lentiviral system was employed to produce viral supernatants. Plasmids (pHR-EF1α_IRES_GFP_SIN backbone) containing MART-1 TCR⁶², Clone 5 TCR⁶³ and 1E6 TCR⁶⁴, in addition to the MART-1 peptide (H3)⁶² were introduced into HEK293T cells using lipofectamine transfection (Lipofectamine 2000, Thermo Fisher). The resulting VSV-G pseudotyped viral supernatants were concentrated by ultracentrifugation and stored at −80° C. until needed.

### Cell Lines and Primary Cultures

K-562 cells transduced with HLA-A2 were kindly provided by Dr. James Riley, University of Pennsylvania, Philadelphia. T2 cell line was purchased from ATCC and cultured according to manufacturer's recommendations. To obtain B and T cells for the experiments, 8 anonymous human donor PBMCs were obtained from the New York Blood Center. PBMCs were isolated by histopaque (Sigma Aldrich) density centrifugation.

The B lymphoblastoid cell line (LCL) was generated by Epstein-Barr virus (EBV) infection of MACS-sorted B cells from the HLA-A2+ PBMC followed by culturing and passaging the cells for 3 months⁶⁵.

The remaining PBMCs were frozen in multiple aliquots for later isolation and transduction of HLA-A2+ primary T cells autologous to the LCLs. On day 0 of each experiment, an aliquot of PBMCs were stimulated with 25 uL/mL of ImmunoCult™ Human CD3/CD28/CD2 T Cell Activator (StemCell Technologies) in X Vivo 15 media (Lonza) supplemented with 10% human serum (Gemini Bio-Products) and the following cytokines: 100 IU/mL IL2, 10 ng/mL IL7, and 10 ng/mL IL15.

### Cell Transduction

Flat-bottom 96-well plates were precoated with retronectin (25 μg/mL, Clontech) for 1 hour at 37° C. Lentiviruses at an MOI of 10-20 were added to the coated wells, followed by centrifugation at 1500g for 90 minutes at 32° C. Subsequently, primary human T cells were added to the wells, followed by another centrifugation at 500g for 10 minutes at 32° C. The cells were then incubated overnight in a CO2 incubator. The following day, the wells were washed, and new medium containing the cytokines IL-2, IL-7, and IL-15 was added. Additionally, irradiated allogeneic PBMCs and LCLs plus PHA were added to the wells to expand the transduced cells.

### CRISPR Mediated TCR Removal

TCR knockout (KO) T cells were generated by electroporating Cas9 ribonucleoproteins (RNPs) on day 2 post-activation, either simultaneously with MART-1 TCR lentivirus (in the C+V group) or alone (in the C then V group). CRISPR RNA (crRNA) sequences were synthesized by Integrated DNA Technologies, Inc., (IDT). Equimolar ratios of crRNA and tracrRNA were heated at 95° C. for 5 minutes to form guide RNA (gRNA). After cooling to room temperature, Cas9 (IDT) was combined with gRNA at an equimolar ratio for 20 minutes at room temperature. The RNP was mixed with Resuspension Buffer R (Thermo Fisher) to a volume of 10 μl and used to resuspend 2×10⁵-3×10⁵ T cells per electroporation reaction; 2 μl of electroporation enhancer (IDT) was then added. Cells were electroporated using the Neon Transfection System under the following parameters: 1600 V voltage, 10 ms width, and 3 pulses.

### Human Fetal Tissues

All human fetal tissues (gestational age 17-21 weeks) were obtained from Advanced Bioscience Resources (Alameda, CA). Fetal liver was prepared as previously described 66. Briefly, single cell suspensions were generated from fetal livers by Liberase digestion (Sigma-Aldrich). CD34+ cells were isolated using positive selection by magnetic-activated cell sorting (MACS) with anti-human CD34+ microbeads according to the manufacturer's instructions (Miltenyi Biotec).

### Mouse Studies

Male and female NSG (NOD.Cg-Prkdcscid Il2rgtm1Wjl/SzJ) and NSG-MHC I/II DKO (NOD.Cg-Prkdcscid H2-K1tm1Bpe H2-Ab1em1Mvw H2-D1tm1Bpe Il2rgtm1Wjl/SzJ) mice aged 6-7 weeks were purchased from Jackson Laboratory and/or bred in-house. The animals were housed in a pathogen-free microisolator environment. All mouse procedures were conducted in accordance with approved protocols from the Institutional Animal Care and Use Committee (IACUC) at Columbia University Medical Center. Mice were surgically thymectomized as described⁶⁷ and allowed to recover for at least two weeks. Mice were then irradiated with 1 Gy total body irradiation (TBI) by an X-Ray irradiator (RS-2000, Rad Source Technologies, Inc.) and transplanted with 1.5-2×10⁵ CD34+ fetal liver cells intravenously. For the K562 HLA-A2+ tumor model, 7- to 10-week-old NSG-MHC 1/II DKO mice were subcutaneously injected with 1×10⁶ tumoral cells suspended in 100 μl of Matrigel (Corning). T cells generated under different conditions were injected intravenously on day 3 following tumor inoculation. For the GvHD study, 7- to 10-week-old NSG mice were injected with T cells via the tail vein. In both studies, T cells were administered at a dose of 2×10⁶ cells per mouse. Mice in the tumor and GvHD experiments were monitored by measuring tumor size and assessing GvHD score, respectively. Additionally, at certain time points, mice were bled, and immune cells in the blood were characterized by flow cytometry.

### Histological Analysis

Mice were sacrificed and tissues were harvested and fixed in 4% formaldehyde for 24-48 hrs. Paraffin embedded sections were cut into 5 uM sections and stained with hematoxylin and eosin (H&E) as described⁶⁸. Images were obtained on Leica SCN 400 (Leica Microsystems, Wetzlar, German) whole slide scanning platform.

### Multi-Color Flow Cytometry Analyses

Samples were treated with mouse and human Fc block (BD Biosciences) along with surface markers of fluorochrome-conjugated antibodies of various combinations. For intracellular staining, cells were permeabilized using the eBioscience™ Foxp3/Transcription Factor Staining Buffer Set (ThermoFisher Scientific) and then stained with fluorochrome-conjugated antibodies (see Table 2) of various combinations.

For processing blood samples, 50 μl of blood per mouse was collected, and red blood cells (RBCs) were lysed in ACK lysis buffer. Following antibody staining, 1 μl of counting beads (123ecount eBeads Counting Beads, ThermoFisher Scientific) was added to each sample to allow quantification of blood cell subsets. Flow cytometry samples were analyzed using either Fortessa (BD Biosciences, Mountain View, CA) or Aurora (Cytek Biosciences, Fremont, CA) instruments. Data analyses were performed using FlowJo software (Tree Star, San Carlos, CA).

### Targeted Locus Amplification

Targeted locus amplification (TLA) technology uses the physical proximity of nucleotides within a locus of interest to generate a map of original sequences and corresponding inserted transgenes³⁶. The cells were frozen and shipped to Cergentis (Utrecht, Netherlands). TLA was then performed as described previously^{36, 69}. Briefly, DNA was crosslinked, fragmented, re-ligated, and de-crosslinked. This product served as the TLA template, which was subsequently fragmented, circularized, and amplified with inverse primers complementary to a short locus-specific sequence. Once the complete locus was amplified, ˜2 kb segments were sheared. Libraries were prepared for sequencing on an Illumina platform. Two sets of primers (see Table 3) targeting the transgenic RRE or GFP sequences were used in individual TLA amplifications

### Statistical Analysis

Analyses were conducted in Graphpad Prism 8.0 or 9.0 (GraphPad Software). Statistics were conducted by Student's T tests or ANOVA and p values<0.05 were considered significant. Data are presented using mean±SEM.

### REFERENCES

1. Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, Rosenberg S A. Cancer regression in patients after transfer of genetically engineered lymphocytes. Science. 2006; 314(5796):126-9. Epub 2006/09/02. doi: 10.1126/science.1129003. PubMed PMID: 16946036; PMCID: PMC2267026.
2. Morotti M, Albukhari A, Alsaadi A, Artibani M, Brenton J D, Curbishley S M, Dong T, Dustin M L, Hu Z, McGranahan N, Miller M L, Santana-Gonzalez L, Seymour L W, Shi T, Van Loo P, Yau C, White H, Wietek N, Church D N, Wedge D C, Ahmed A A. Promises and challenges of adoptive T-cell therapies for solid tumours. Br J Cancer. 2021; 124(11):1759-76. Epub 2021/03/31. doi: 10.1038/s41416-021-01353-6. PubMed PMID: 33782566; PMCID: PMC8144577.
3. Perica K, Varela J C, Oelke M, Schneck J. Adoptive T cell immunotherapy for cancer. Rambam Maimonides Med J. 2015; 6(1):e0004. Epub 2015/02/27. doi: 10.5041/RMMJ.10179. PubMed PMID: 25717386; PMCID: PMC4327320.
4. Rapoport A P, Stadtmauer E A, Binder-Scholl G K, Goloubeva O, Vogl D T, Lacey S F, Badros A Z, Garfall A, Weiss B, Finklestein J, Kulikovskaya I, Sinha S K, Kronsberg S, Gupta M, Bond S, Melchiori L, Brewer J E, Bennett A D, Gerry A B, Pumphrey N J, Williams D, Tayton-Martin H K, Ribeiro L, Holdich T, Yanovich S, Hardy N, Yared J, Kerr N, Philip S, Westphal S, Siegel D L, Levine B L, Jakobsen B K, Kalos M, June C H. N Y-ESO-1-specific TCR-engineered T cells mediate sustained antigen-specific antitumor effects in myeloma. Nat Med. 2015; 21(8):914-21. Epub 20150720. doi: 10.1038/nm.3910. PubMed PMID: 26193344; PMCID: PMC4529359.
5. Robbins P F, Kassim S H, Tran T L, Crystal J S, Morgan R A, Feldman S A, Yang J C, Dudley M E, Wunderlich J R, Sherry R M, Kammula U S, Hughes M S, Restifo N P, Raffeld M, Lee C C, Li Y F, El-Gamil M, Rosenberg S A. A pilot trial using lymphocytes genetically engineered with an NY-ESO-1-reactive T-cell receptor: long-term follow-up and correlates with response. Clin Cancer Res. 2015; 21(5):1019-27. Epub 20141223. doi: 10.1158/1078-0432.CCR-14-2708. PubMed PMID: 25538264; PMCID: PMC4361810.
6. Deng Q, Han G, Puebla-Osorio N, Ma M C J, Strati P, Chasen B, Dai E, Dang M, Jain N, Yang H, Wang Y, Zhang S, Wang R, Chen R, Showell J, Ghosh S, Patchva S, Zhang Q, Sun R, Hagemeister F, Fayad L, Samaniego F, Lee H C, Nastoupil L J, Fowler N, Eric Davis R, Westin J, Neelapu S S, Wang L, Green M R. Characteristics of anti-CD19 CAR T cell infusion products associated with efficacy and toxicity in patients with large B cell lymphomas. Nat Med. 2020; 26(12):1878-87. Epub 20201005. doi: 10.1038/s41591-020-1061-7. PubMed PMID: 33020644; PMCID: PMC8446909.
7. Fraietta J A, Lacey S F, Orlando E J, Pruteanu-Malinici I, Gohil M, Lundh S, Boesteanu A C, Wang Y, O'Connor R S, Hwang W T, Pequignot E, Ambrose D E, Zhang C, Wilcox N, Bedoya F, Dorfmeier C, Chen F, Tian L, Parakandi H, Gupta M, Young R M, Johnson F B, Kulikovskaya I, Liu L, Xu J, Kassim S H, Davis M M, Levine B L, Frey N V, Siegel D L, Huang A C, Wherry E J, Bitter H, Brogdon J L, Porter D L, June C H, Melenhorst J J. Determinants of response and resistance to CD19 chimeric antigen receptor (CAR) T cell therapy of chronic lymphocytic leukemia. Nat Med. 2018; 24(5):563-71. Epub 20180430. doi: 10.1038/s41591-018-0010-1. PubMed PMID: 29713085; PMCID: PMC6117613.
8. Das R K, Vernau L, Grupp S A, Barrett D M. Naive T-cell Deficits at Diagnosis and after Chemotherapy Impair Cell Therapy Potential in Pediatric Cancers. Cancer Discov. 2019; 9(4):492-9. Epub 20190110. doi: 10.1158/2159-8290.CD-18-1314. PubMed PMID: 30630850; PMCID: PMC6676489.
9. Graham C E, Jozwik A, Quartey-Papafio R, Ioannou N, Metelo A M, Scala C, Dickson G, Stewart O, Almena-Carrasco M, Peranzoni E, Ramsay A G, Patten P E M, Pertel T, Farzaneh F, Dupouy S, Pepper A, Benjamin R. Gene-edited healthy donor CAR T cells show superior anti-tumour activity compared to CAR T cells derived from patients with lymphoma in an in vivo model of high-grade lymphoma. Leukemia. 2021; 35(12):3581-4. Epub 20210618. doi: 10.1038/s41375-021-01324-z. PubMed PMID: 34145373; PMCID: PMC8632681.
10. Maude S L, Laetsch T W, Buechner J, Rives S, Boyer M, Bittencourt H, Bader P, Verneris M R, Stefanski H E, Myers G D, Qayed M, De Moerloose B, Hiramatsu H, Schlis K, Davis K L, Martin P L, Nemecek E R, Yanik G A, Peters C, Baruchel A, Boissel N, MechinaudF, Balduzzi A, Krueger J, June C H, Levine B L, Wood P, Taran T, Leung M, Mueller K T, Zhang Y, Sen K, Lebwohl D, Pulsipher M A, Grupp S A. Tisagenlecleucel in Children and Young Adults with B-Cell Lymphoblastic Leukemia. N Engl J Med. 2018; 378(5):439-48. doi: 10.1056/NEJMoa1709866. PubMed PMID: 29385370; PMCID: PMC5996391.
11. Neelapu S S, Locke F L, Bartlett N L, Lekakis L J, Miklos D B, Jacobson C A, Braunschweig I, Oluwole 00, Siddiqi T, Lin Y, Timmerman J M, Stiff P J, Friedberg J W, Flinn I W, Goy A, Hill B T, Smith M R, Deol A, Farooq U, McSweeney P, Munoz J, Avivi I, Castro J E, Westin J R, Chavez J C, Ghobadi A, Komanduri K V, Levy R, Jacobsen E D, Witzig T E, Reagan P, Bot A, Rossi J, Navale L, Jiang Y, Aycock J, Elias M, Chang D, Wiezorek J, Go W Y. Axicabtagene Ciloleucel CAR T-Cell Therapy in Refractory Large B-Cell Lymphoma. N Engl J Med. 2017; 377(26):2531-44. Epub 20171210. doi: 10.1056/NEJMoa1707447. PubMed PMID: 29226797; PMCID: PMC5882485.
12. Morton L T, Reijmers R M, Wouters A K, Kweekel C, Remst D F G, Pothast C R, Falkenburg J H F, Heemskerk M H M. Simultaneous Deletion of Endogenous TCRalphabeta for TCR Gene Therapy Creates an Improved and Safe Cellular Therapeutic. Mol Ther. 2020; 28(1):64-74. Epub 2019/10/23. doi: 10.1016/j.ymthe.2019.10.001. PubMed PMID: 31636040; PMCID: PMC6953894.
13. Okamoto S, Mineno J, Ikeda H, Fujiwara H, Yasukawa M, Shiku H, Kato I. Improved expression and reactivity of transduced tumor-specific TCRs in human lymphocytes by specific silencing of endogenous TCR. Cancer Res. 2009; 69(23):9003-11. Epub 2009/11/12. doi: 10.1158/0008-5472.CAN-09-1450. PubMed PMID: 19903853.
14. Tsimberidou A M, Van Morris K, Vo H H, Eck S, Lin Y F, Rivas J M, Andersson B S. T-cell receptor-based therapy: an innovative therapeutic approach for solid tumors. J Hematol Oncol. 2021; 14(1):102. Epub 20210630. doi: 10.1186/s13045-021-01115-0. PubMed PMID: 34193217; PMCID: PMC8243554.
15. Jahn L, Hombrink P, Hagedoorn R S, Kester M G, van der Steen D M, Rodriguez T, Pentcheva-Hoang T, de Ru A H, Schoonakker M P, Meeuwsen M H, Griffioen M, van Veelen P A, Falkenburg J H, Heemskerk M H. TCR-based therapy for multiple myeloma and other B-cell malignancies targeting intracellular transcription factor BOB1. Blood. 2017; 129(10):1284-95. Epub 2017/01/06. doi: 10.1182/blood-2016-09-737536. PubMed PMID: 28053195.
16. Liu Y, Yan X, Zhang F, Zhang X, Tang F, Han Z, Li Y. TCR-T Immunotherapy: The Challenges and Solutions. Front Oncol. 2021; 11:794183. Epub 20220125. doi: 10.3389/fonc.2021.794183. PubMed PMID: 35145905; PMCID: PMC8822241.
17. Rath J A, Arber C. Engineering Strategies to Enhance TCR-Based Adoptive T Cell Therapy. Cells. 2020; 9(6). Epub 20200618. doi: 10.3390/cells9061485. PubMed PMID: 32570906; PMCID: PMC7349724.
18. Bendle G M, Linnemann C, Hooijkaas A L, Bies L, de Witte M A, Jorritsma A, Kaiser A D, Pouw N, Debets R, Kieback E, Uckert W, Song J Y, Haanen J B, Schumacher T N. Lethal graft-versus-host disease in mouse models of T cell receptor gene therapy. Nat Med. 2010; 16(5):565-70, 1p following 70. Epub 2010/04/20. doi: 10.1038/nm.2128. PubMed PMID: 20400962.
19. van Loenen M M, de Boer R, Amir A L, Hagedoorn R S, Volbeda G L, Willemze R, van Rood J J, Falkenburg J H, Heemskerk M H. Mixed T cell receptor dimers harbor potentially harmful neoreactivity. Proc Natl Acad Sci USA. 2010; 107(24):10972-7. Epub 2010/06/11. doi: 10.1073/pnas.1005802107. PubMed PMID: 20534461; PMCID: PMC2890759.
20. Alarcon B, Berkhout B, Breitmeyer J, Terhorst C. Assembly of the human T cell receptor-CD3 complex takes place in the endoplasmic reticulum and involves intermediary complexes between the CD3-gamma.delta.epsilon core and single T cell receptor alpha or beta chains. J Biol Chem. 1988; 263(6):2953-61. PubMed PMID: 2963821.
21. Ahmadi M, King J W, Xue S A, Voisine C, Holler A, Wright G P, Waxman J, Morris E, Stauss H J. CD3 limits the efficacy of TCR gene therapy in vivo. Blood. 2011; 118(13):3528-37. Epub 2011/07/14. doi: 10.1182/blood-2011-04-346338. PubMed PMID: 21750319.
22. Heemskerk M H, Hagedoorn R S, van der Hoorn M A, van der Veken L T, Hoogeboom M, Kester M G, Willemze R, Falkenburg J H. Efficiency of T-cell receptor expression in dual-specific T cells is controlled by the intrinsic qualities of the TCR chains within the TCR-CD3 complex. Blood. 2007; 109(1):235-43. Epub 2006/09/14. doi: 10.1182/blood-2006-03-013318. PubMed PMID: 16968899.
23. Li Y, Moysey R, Molloy P E, Vuidepot A L, Mahon T, Baston E, Dunn S, Liddy N, Jacob J, Jakobsen B K, Boulter J M. Directed evolution of human T-cell receptors with picomolar affinities by phage display. Nat Biotechnol. 2005; 23(3):349-54. Epub 2005/02/22. doi: 10.1038/nbt1070. PubMed PMID: 15723046.
24. Govers C, Sebestyen Z, Coccoris M, Willemsen R A, Debets R. T cell receptor gene therapy: strategies for optimizing transgenic TCR pairing. Trends Mol Med. 2010; 16(2):77-87. Epub 2010/02/04. doi: 10.1016/j.molmed.2009.12.004. PubMed PMID: 20122868.
25. Legut M, Dolton G, Mian A A, Ottmann O G, Sewell A K. CRISPR-mediated TCR replacement generates superior anticancer transgenic T cells. Blood. 2018; 131(3):311-22. Epub 2017/11/11. doi: 10.1182/blood-2017-05-787598. PubMed PMID: 29122757; PMCID: PMC5774207.
26. Mastaglio S, Genovese P, Magnani Z, Ruggiero E, Landoni E, Camisa B, Schiroli G, Provasi E, Lombardo A, Reik A, Cieri N, Rocchi M, Oliveira G, Escobar G, Casucci M, Gentner B, Spinelli A, Mondino A, Bondanza A, Vago L, Ponzoni M, Ciceri F, Holmes M C, Naldini L, Bonini C. NY-ESO-1 TCR single edited stem and central memory T cells to treat multiple myeloma without graft-versus-host disease. Blood. 2017; 130(5):606-18. Epub 2017/06/24. doi: 10.1182/blood-2016-08-732636. PubMed PMID: 28637663.
27. Schober K, Muller T R, Gokmen F, Grassmann S, Effenberger M, Poltorak M, Stemberger C, Schumann K, Roth T L, Marson A, Busch D H. Orthotopic replacement of T-cell receptor alpha- and beta-chains with preservation of near-physiological T-cell function. Nat Biomed Eng. 2019; 3(12):974-84. Epub 2019/06/12. doi: 10.1038/s41551-019-0409-0. PubMed PMID: 31182835.
28. Roth T L, Puig-Saus C, Yu R, Shifrut E, Carnevale J, Li P J, Hiatt J, Saco J, Krystofinski P, Li H, Tobin V, Nguyen D N, Lee M R, Putnam A L, Ferris A L, Chen J W, Schickel J N, Pellerin L, Carmody D, Alkorta-Aranburu G, Del Gaudio D, Matsumoto H, Morell M, Mao Y, Cho M, Quadros R M, Gurumurthy C B, Smith B, Haugwitz M, Hughes S H, Weissman J S, Schumann K, Esensten J H, May A P, Ashworth A, Kupfer G M, Greeley S A W, Bacchetta R, Meffre E, Roncarolo M G, Romberg N, Herold K C, Ribas A, Leonetti M D, Marson A. Reprogramming human T cell function and specificity with non-viral genome targeting. Nature. 2018; 559(7714):405-9. Epub 2018/07/12. doi: 10.1038/s41586-018-0326-5. PubMed PMID: 29995861; PMCID: PMC6239417.
29. Verkuijl S A, Rots M G. The influence of eukaryotic chromatin state on CRISPR-Cas9 editing efficiencies. Curr Opin Biotechnol. 2019; 55:68-73. Epub 2018/09/07. doi: 10.1016/j.copbio.2018.07.005. PubMed PMID: 30189348.
30. Chen Y T, Stockert E, Jungbluth A, Tsang S, Coplan K A, Scanlan M J, Old U. Serological analysis of Melan-A(MART-1), a melanocyte-specific protein homogeneously expressed in human melanomas. Proc Natl Acad Sci USA. 1996; 93(12):5915-9. Epub 1996/06/11. doi: 10.1073/pnas.93.12.5915. PubMed PMID: 8650193; PMCID: PMC39162.
31. Hosken N A, Bevan M J. Defective presentation of endogenous antigen by a cell line expressing class I molecules. Science. 1990; 248(4953):367-70. Epub 1990/04/20. doi: 10.1126/science.2326647. PubMed PMID: 2326647.
32. Drew S I, Terasaki P I, Billing R J, Bergh O J, Minowada J, Klein E. Group-specific human granulocyte antigens on a chronic myelogenous leukemia cell line with a Philadelphia chromosome marker. Blood. 1977; 49(5):715-8. Epub 1977/05/01. PubMed PMID: 66956.
33. Brehm M A, Kenney L L, Wiles M V, Low B E, Tisch R M, Burzenski L, Mueller C, Greiner D L, Shultz L D. Lack of acute xenogeneic graft-versus-host disease, but retention of T-cell function following engraftment of human peripheral blood mononuclear cells in NSG mice deficient in MHC class I and II expression. FASEB J. 2019; 33(3):3137-51. Epub 20181101. doi: 10.1096/fj.201800636R. PubMed PMID: 30383447; PMCID: PMC6404556.
34. Matrai J, Cantore A, Bartholomae C C, Annoni A, Wang W, Acosta-Sanchez A, Samara-Kuko E, De Waele L, Ma L, Genovese P, Damo M, Arens A, Goudy K, Nichols T C, von Kalle C, MK L C, Roncarolo M G, Schmidt M, Vandendriessche T, Naldini L. Hepatocyte-targeted expression by integrase-defective lentiviral vectors induces antigen-specific tolerance in mice with low genotoxic risk. Hepatology. 2011; 53(5):1696-707. doi: 10.1002/hep.24230. PubMed PMID: 21520180; PMCID: PMC3112259.
35. Riepe C, Zelin E, Frankino P A, Meacham Z A, Fernandez S G, Ingolia N T, Corn J E. Double stranded DNA breaks and genome editing trigger loss of ribosomal protein RPS27A. FEBS J. 2022; 289(11):3101-14. Epub 20220110. doi: 10.1111/febs.16321. PubMed PMID: 34914197; PMCID: PMC9295824.
36. de Vree P J, de Wit E, Yilmaz M, van de Heijning M, Klous P, Verstegen M J, Wan Y, Teunissen H, Krijger P H, Geeven G, Eijk P P, Sie D, Ylstra B, Hulsman L O, van Dooren M F, van Zutven L J, van den Ouweland A, Verbeek S, van Dijk K W, Cornelissen M, Das A T, Berkhout B, Sikkema-Raddatz B, van den Berg E, van der Vlies P, Weening D, den Dunnen J T, Matusiak M, Lamkanfi M, Ligtenberg M J, ter Brugge P, Jonkers J, Foekens J A, Martens J W, van der Luijt R, van Amstel H K, van Min M, Splinter E, de Laat W. Targeted sequencing by proximity ligation for comprehensive variant detection and local haplotyping. Nat Biotechnol. 2014; 32(10):1019-25. Epub 20140817. doi: 10.1038/nbt.2959. PubMed PMID: 25129690.
37. Waldman A D, Fritz J M, Lenardo M J. A guide to cancer immunotherapy: from T cell basic science to clinical practice. Nat Rev Immunol. 2020; 20(11):651-68. Epub 20200520. doi: 10.1038/s41577-020-0306-5. PubMed PMID: 32433532; PMCID: PMC7238960.
38. Hayes C. Cellular immunotherapies for cancer. Ir J Med Sci. 2021; 190(1):41-57. Epub 20200701. doi: 10.1007/si1845-020-02264-w. PubMed PMID: 32607912; PMCID: PMC7326625.
39. Chandran S S, Klebanoff C A. T cell receptor-based cancer immunotherapy: Emerging efficacy and pathways of resistance. Immunol Rev. 2019; 290(1):127-47. doi: 10.1111/imr.12772. PubMed PMID: 31355495; PMCID: PMC7027847.
40. Depil S, Duchateau P, Grupp S A, Mufti G, Poirot L. ‘Off-the-shelf’ allogeneic CAR T cells: development and challenges. Nat Rev Drug Discov. 2020; 19(3):185-99. Epub 20200103. doi: 10.1038/s41573-019-0051-2. PubMed PMID: 31900462.
41. Bialer G, Horovitz-Fried M, Ya'acobi S, Morgan R A, Cohen C J. Selected murine residues endow human TCR with enhanced tumor recognition. J Immunol. 2010; 184(11):6232-41. Epub 20100428. doi: 10.4049/jimmunol.0902047. PubMed PMID: 20427762.
42. Cohen C J, Zhao Y, Zheng Z, Rosenberg S A, Morgan R A. Enhanced antitumor activity of murine-human hybrid T-cell receptor (TCR) in human lymphocytes is associated with improved pairing and TCR/CD3 stability. Cancer Res. 2006; 66(17):8878-86. doi: 10.1158/0008-5472.CAN-06-1450. PubMed PMID: 16951205; PMCID: PMC2147082.
43. Cohen C J, Li Y F, El-Gamil M, Robbins P F, Rosenberg S A, Morgan R A. Enhanced antitumor activity of T cells engineered to express T-cell receptors with a second disulfide bond. Cancer Res. 2007; 67(8):3898-903. doi: 10.1158/0008-5472.CAN-06-3986. PubMed PMID: 17440104; PMCID: PMC2147081.
44. Kuball J, Dossett M L, Wolfl M, Ho W Y, Voss R H, Fowler C, Greenberg P D. Facilitating matched pairing and expression of TCR chains introduced into human T cells. Blood. 2007; 109(6):2331-8. Epub 20061102. doi: 10.1182/blood-2006-05-023069. PubMed PMID: 17082316; PMCID: PMC1852191.
45. Scholten K B, Kramer D, Kueter E W, Graf M, Schoedl T, Meijer C J, Schreurs M W, Hooijberg E. Codon modification of T cell receptors allows enhanced functional expression in transgenic human T cells. Clin Immunol. 2006; 119(2):135-45. Epub 20060202. doi: 10.1016/j.clim.2005.12.009. PubMed PMID: 16458072.
46. Provasi E, Genovese P, Lombardo A, Magnani Z, Liu P Q, Reik A, Chu V, Paschon D E, Zhang L, Kuball J, Camisa B, Bondanza A, Casorati G, Ponzoni M, Ciceri F, Bordignon C, Greenberg P D, Holmes M C, Gregory P D, Naldini L, Bonini C. Editing T cell specificity towards leukemia by zinc finger nucleases and lentiviral gene transfer. Nat Med. 2012; 18(5):807-15. doi: 10.1038/nm.2700. PubMed PMID: 22466705; PMCID: PMC5019824.
47. Berdien B, Mock U, Atanackovic D, Fehse B. TALEN-mediated editing of endogenous T-cell receptors facilitates efficient reprogramming of T lymphocytes by lentiviral gene transfer. Gene Ther. 2014; 21(6):539-48. Epub 20140327. doi: 10.1038/gt.2014.26. PubMed PMID: 24670996.
48. Gonzalez Castro N, Bjelic J, Malhotra G, Huang C, Alsaffar S H. Comparison of the Feasibility, Efficiency, and Safety of Genome Editing Technologies. Int J Mol Sci. 2021; 22(19). Epub 20210926. doi: 10.3390/ijms221910355. PubMed PMID: 34638696; PMCID: PMC8509008.
49. Osborn M J, Webber B R, Knipping F, Lonetree C L, Tennis N, DeFeo A P, McElroy A N, Starker C G, Lee C, Merkel S, Lund T C, Kelly-Spratt K S, Jensen M C, Voytas D F, von Kalle C, Schmidt M, Gabriel R, Hippen K L, Miller J S, Scharenberg A M, Tolar J, Blazar B R. Evaluation of TCR Gene Editing Achieved by TALENs, CRISPR/Cas9, and megaTAL Nucleases. Mol Ther. 2016; 24(3):570-81. Epub 20151027. doi: 10.1038/mt.2015.197. PubMed PMID: 26502778; PMCID: PMC4786913.
50. Kagoya Y, Guo T, Yeung B, Saso K, Anczurowski M, Wang C H, Murata K, Sugata K, Saijo H, Matsunaga Y, Ohashi Y, Butler M O, Hirano N. Genetic Ablation of HLA Class I, Class II, and the T-cell Receptor Enables Allogeneic T Cells to Be Used for Adoptive T-cell Therapy. Cancer Immunol Res. 2020; 8(7):926-36. Epub 20200422. doi: 10.1158/2326-6066.CIR-18-0508. PubMed PMID: 32321775.
51. Ren J, Liu X, Fang C, Jiang S, June C H, Zhao Y. Multiplex Genome Editing to Generate Universal CAR T Cells Resistant to PD1 Inhibition. Clin Cancer Res. 2017; 23(9):2255-66. Epub 20161104. doi: 10.1158/1078-0432.CCR-16-1300. PubMed PMID: 27815355; PMCID: PMC5413401.
52. Benjamin R, Jain N, Maus M V, Boissel N, Graham C, Jozwik A, Yallop D, Konopleva M, Frigault M J, Teshima T, Kato K, Boucaud F, Balandraud S, Gianella-Borradori A, Binlich F, Marchiq I, Dupouy S, Almena-Carrasco M, Pannaux M, Fouliard S, Brissot E, Mohty M, Group C S. UCART19, a first-in-class allogeneic anti-CD19 chimeric antigen receptor T-cell therapy for adults with relapsed or refractory B-cell acute lymphoblastic leukaemia (CALM): a phase 1, dose-escalation trial. Lancet Haematol. 2022; 9(11):e833-e43. Epub 20221010. doi: 10.1016/S2352-3026(22)00245-9. PubMed PMID: 36228643.
53. Qasim W, Zhan H, Samarasinghe S, Adams S, Amrolia P, Stafford S, Butler K, Rivat C, Wright G, Somana K, Ghorashian S, Pinner D, Ahsan G, Gilmour K, Lucchini G, Inglott S, Mifsud W, Chiesa R, Peggs K S, Chan L, Farzeneh F, Thrasher A J, Vora A, Pule M, Veys P. Molecular remission of infant B-ALL after infusion of universal TALEN gene-edited CAR T cells. Sci Transl Med. 2017; 9(374). doi: 10.1126/scitranslmed.aaj2013. PubMed PMID: 28123068.
54. Mitchell R S, Beitzel B F, Schroder A R, Shinn P, Chen H, Berry C C, Ecker J R, Bushman F D. Retroviral DNA integration: ASLV, HIV, and MLV show distinct target site preferences. PLoS Biol. 2004; 2(8):E234. Epub 20040817. doi: 10.1371/journal.pbio.0020234. PubMed PMID: 15314653; PMCID: PMC509299.
55. Schroder A R, Shinn P, Chen H, Berry C, Ecker J R, Bushman F. HIV-1 integration in the human genome favors active genes and local hotspots. Cell. 2002; 110(4):521-9. doi: 10.1016/s0092-8674(02)00864-4. PubMed PMID: 12202041.
56. Shao L, Shi R, Zhao Y, Liu H, Lu A, Ma J, Cai Y, Fuksenko T, Pelayo A, Shah N N, Kochenderfer J N, Norberg S M, Hinrichs C, Highfill S L, Somerville R P, Panch S R, Jin P, Stroncek D F. Genome-wide profiling of retroviral DNA integration and its effect on clinical pre-infusion CAR T-cell products. J Transl Med. 2022; 20(1):514. Epub 20221108. doi: 10.1186/s12967-022-03729-5. PubMed PMID: 36348415; PMCID: PMC9644589.
57. Fraietta J A, Nobles C L, Sammons M A, Lundh S, Carty S A, Reich T J, Cogdill A P, Morrissette J J D, DeNizio J E, Reddy S, Hwang Y, Gohil M, Kulikovskaya I, Nazimuddin F, Gupta M, Chen F, Everett J K, Alexander K A, Lin-Shiao E, Gee M H, Liu X, Young R M, Ambrose D, Wang Y, Xu J, Jordan M S, Marcucci K T, Levine B L, Garcia K C, Zhao Y, Kalos M, Porter D L, Kohli R M, Lacey S F, Berger S L, Bushman F D, June C H, Melenhorst J J. Disruption of TET2 promotes the therapeutic efficacy of CD19-targeted T cells. Nature. 2018; 558(7709):307-12. Epub 20180530. doi: 10.1038/s41586-018-0178-z. PubMed PMID: 29849141; PMCID: PMC6320248.
58. Shah N N, Qin H, Yates B, Su L, Shalabi H, Raffeld M, Ahlman M A, Stetler-Stevenson M, Yuan C, Guo S, Liu S, Hughes S H, Fry T J, Wu X. Clonal expansion of CAR T cells harboring lentivector integration in the CBL gene following anti-CD22 CAR T-cell therapy. Blood Adv. 2019; 3(15):2317-22. doi: 10.1182/bloodadvances.2019000219. PubMed PMID: 31387880; PMCID: PMC6693002.
59. Chavez M, Rane D A, Chen X, Qi L S. Stable expression of large transgenes via the knock-in of an integrase-deficient lentivirus. Nat Biomed Eng. 2023; 7(5):661-71. Epub 20230501. doi: 10.1038/s41551-023-01037-x. PubMed PMID: 37127707.
60. Shy B R, Vykunta V S, Ha A, Talbot A, Roth T L, Nguyen D N, Pfeifer W G, Chen Y Y, Blaeschke F, Shifrut E, Vedova S, Mamedov M R, Chung J J, Li H, Yu R, Wu D, Wolf J, Martin T G, Castro C E, Ye L, Esensten J H, Eyquem J, Marson A. High-yield genome engineering in primary cells using a hybrid ssDNA repair template and small-molecule cocktails. Nat Biotechnol. 2023; 41(4):521-31. Epub 20220825. doi: 10.1038/s41587-022-01418-8. PubMed PMID: 36008610; PMCID: PMC10065198.
61. Schumann K, Lin S, Boyer E, Simeonov D R, Subramaniam M, Gate R E, Haliburton G E, Ye C J, Bluestone J A, Doudna J A, Marson A. Generation of knock-in primary human T cells using Cas9 ribonucleoproteins. Proc Natl Acad Sci USA. 2015; 112(33):10437-42. Epub 20150727. doi: 10.1073/pnas.1512503112. PubMed PMID: 26216948; PMCID: PMC4547290.
62. Li Y, Teteloshvili N, Tan S, Rao S, Han A, Yang Y G, Creusot R J. Humanized Mice Reveal New Insights Into the Thymic Selection of Human Autoreactive CD8(+) T Cells. Front Immunol. 2019; 10:63. Epub 20190204. doi: 10.3389/fimmu.2019.00063. PubMed PMID: 30778347; PMCID: PMC6369192.
63. Tan S, Li Y, Xia J, Jin C H, Hu Z, Duinkerken G, Li Y, Khosravi Maharlooei M, Chavez E, Nauman G, Danzl N, Nakayama M, Roep B O, Sykes M, Yang Y G. Type 1 diabetes induction in humanized mice. Proc Natl Acad Sci USA. 2017; 114(41):10954-9. Epub 20170905. doi: 10.1073/pnas.1710415114. PubMed PMID: 28874533; PMCID: PMC5642714.
64. Skowera A, Ellis R J, Varela-Calvino R, Arif S, Huang G C, Van-Krinks C, Zaremba A, Rackham C, Allen J S, Tree T I, Zhao M, Dayan C M, Sewell A K, Unger W W, Drijfhout J W, Ossendorp F, Roep B O, Peakman M. CTLs are targeted to kill beta cells in patients with type 1 diabetes through recognition of a glucose-regulated preproinsulin epitope. J Clin Invest. 2008; 118(10):3390-402. doi: 10.1172/JCI35449. PubMed PMID: 18802479; PMCID: PMC2542849.
65. Amoli M M, Carthy D, Platt H, Ollier W E. EBV Immortalization of human B lymphocytes separated from small volumes of cryo-preserved whole blood. Int J Epidemiol. 2008; 37 Suppl 1:i41-5. doi: 10.1093/ije/dym285. PubMed PMID: 18381392.
66. Lan P, Tonomura N, Shimizu A, Wang S, Yang Y G. Reconstitution of a functional human immune system in immunodeficient mice through combined human fetal thymus/liver and CD34+ cell transplantation. Blood. 2006; 108(2):487-92. Epub 20060112. doi: 10.1182/blood-2005-11-4388. PubMed PMID: 16410443.
67. Khosravi-Maharlooei M, Hoelzl M, Li H W, Madley R C, Waffarn E E, Danzl N M, Sykes M. Rapid thymectomy of NSG mice to analyze the role of native and grafted thymi in humanized mice. Eur J Immunol. 2020; 50(1):138-41. Epub 20191128. doi: 10.1002/eji.201948205. PubMed PMID: 31583677; PMCID: PMC6940512.
68. Khosravi-Maharlooei M, Pakyari M, Jalili R B, Kilani R T, Ghahary A. Intraperitoneal injection of IDO-expressing dermal fibroblasts improves the allograft survival. Clin Immunol. 2017; 174:1-9. Epub 20161029. doi: 10.1016/j.clim.2016.10.012. PubMed PMID: 27989895.
69. Hottentot Q P, van Min M, Splinter E, White S J. Targeted Locus Amplification and Next-Generation Sequencing. Methods Mol Biol. 2017; 1492:185-96. doi: 10.1007/978-1-4939-6442-0_13. PubMed PMID: 27822865.

1. A method of treating a disease in a subject comprising administering an adoptive cell therapy (ACT) to the subject, the ACT comprising delivering a modified lymphocyte comprising
i) a modified T cell receptor (TCR) or a chimeric antigen receptor (CAR);
ii) at least one inactivated endogenous TCRα chain allele; and
iii) at least one inactivated endogenous TCRβ chain allele;
thereby treating the disease of the subject.

2. The method of claim 1, wherein the lymphocyte comprises a modified TCR, and the modified TCR comprises a TCRα chain expressed from an exogenous nucleotide coding sequence lacking introns and a TCRβ chain expressed from an exogenous nucleotide coding sequence lacking introns.

3. The method of claim 1, wherein each endogenous TCRα chain allele is inactivated in the modified lymphocyte, preferably such that the inactivation knocks out each TCRα chain allele, preferably such that an endogenous, full-length and/or functional TCRα chain is not expressed by the modified lymphocyte.

4. The method of claim 1, wherein each endogenous TCRβ chain allele is inactivated in the modified lymphocyte, preferably such that the inactivation knocks out each TCRβ chain allele, preferably such that an endogenous, full-length and/or functional TCRβ chain is not expressed by the modified lymphocyte.

5. The method of claim 1, wherein the inactivated endogenous TCRα chain allele is a T Cell Receptor Alpha Constant (TRAC) allele, preferably wherein each TRAC allele is inactivated in the modified lymphocyte.

6. The method of claim 1, wherein the inactivated endogenous TCRα chain allele is an inactivated endogenous TRAC allele comprising genetic modification within 50 nucleotides of a TRAC intron-exon boundary.

7. The method of claim 6, wherein the TRAC intron-exon boundary is the boundary between (i) TRAC intron 1 and exon 1, (ii) TRAC exon 1 and intron 2, (iii) TRAC intron 2 and exon 2, (iv) TRAC exon 2 and intron 3, (v) TRAC intron 3 and exon 3, and/or (vi) TRAC exon 3 and intron 4, preferably between TRAC intron 1 and exon 1 and/or between TRAC exon 1 and intron 2.

8. The method of claim 1, wherein the inactivated endogenous TCRα chain allele comprises a genetic modification to a sequence comprising CAGGGTTCTGGATATCTGT (SEQ ID NO: 3), CTGCCCTTACCTGGGCT (SEQ ID NO: 4), CATCACAGGAACTTTCTAAA (SEQ ID NO: 5), AGCTTTGAAACAGGTAAGAC (SEQ ID NO: 6), TTCGTATCTGTAAAACCAAG (SEQ ID NO: 7), and/or TCAAGGCCCCTCACCTCAGC (SEQ ID NO: 8) located on a TRAC coding or template strand.

9. The method of claim 1, wherein the inactivated endogenous TCRβ chain allele is a T Cell Receptor Beta Constant 1 (TRBC1) allele or a T Cell Receptor Beta Constant 2 (TRBC2) allele, preferably wherein each TRBC1 allele and each TRBC2 allele is inactivated in the modified lymphocyte.

10. The method of claim 1, wherein the inactivated endogenous TCRβ chain allele is an inactivated endogenous TRBC1 allele and/or an inactivated endogenous TRBC2 allele comprising a genetic modification within 50 nucleotides of a TRBC1 intron-exon boundary and/or a TRBC2 intron-exon boundary.

11. The method of claim 10, wherein the TRBC1 intron-exon boundary is the boundary between TRBC1 exon 1 and intron 2, and/or wherein the TRBC2 intron-exon boundary is the boundary between TRBC2 exon 1 and intron 2.

12. The method of claim 1, wherein the inactivated endogenous TCRβ chain allele comprises a genetic modification to a sequence comprising CCTGGGGTAGAGCAGGTGAG (SEQ ID NO: 1) and/or CCACTCACCTGCTCTACCCC (SEQ ID NO: 2) located on a TRBC1 coding or template strand and/or a TRBC2 coding or template strand.

13. The method of claim 1, wherein the modified T cell receptor is encoded by a codon-optimized nucleotide sequence or a non-codon-optimized nucleotide sequence.

14. The method of claim 1, wherein a portion of the inactivated endogenous TCRα chain allele and/or inactivated endogenous TCRβ chain allele is excised, and/or comprises at least two genetic modifications.

15. The method of claim 1, wherein the modified TCR is a transgenic TCR and/or an exogenously introduced TCR.

16. The method of claim 1, wherein the modified TCR is exogenously introduced into the lymphocyte by transduction of a viral vector, preferably a lentiviral vector, or by delivery of a naked DNA molecule.

17. A method of modifying a cell for adoptive cell therapy, the method comprising:
inactivating an endogenous TCRα chain allele and an endogenous TCRβ chain allele encoded by the cell, preferably by introducing at least one engineered nuclease to the cell,
thereby modifying the cell for adoptive cell therapy.

18. The method of claim 17, wherein each endogenous TCRα chain allele is inactivated in the modified cell, preferably such that the inactivating knocks out each TCRα chain allele, preferably such that an endogenous, full-length and/or functional TCRα chain is not expressed by the cell and/or wherein each endogenous TCRβ chain allele is inactivated in the modified cell, preferably such that the inactivating knocks out each TCRβ chain allele, preferably such that an endogenous, full-length and/or functional TCRβ chain is not expressed by the cell.

19. A mammalian immune cell modified by having a modified T cell receptor (TCR) or a chimeric antigen receptor (CAR); at least one inactivated endogenous TCRα chain allele; and at least one inactivated endogenous TCRβ chain allele.

20. A method for reducing the likelihood of a graft versus host disease associated with a TCR or CAR-T therapy for a pathology in a subject, comprising administering to the subject a modified lymphocyte of claim 19 comprising
i) a modified T cell receptor (TCR) or a chimeric antigen receptor (CAR);
ii) at least one inactivated endogenous TCRα chain allele; and
iii) at least one inactivated endogenous TCRβ chain allele;
thereby treating the pathology in the subject and reducing the likelihood of a graft versus host disease in the subject.
or
a method for enhancing expression and/or formation of a therapeutic TCR in a cell modified for an adoptive cell therapy, the method comprising:
inactivating an endogenous TCRα chain allele and an endogenous TCRβ chain allele encoded by the cell, preferably by introducing at least one engineered nuclease to the cell and expressing the therapeutic TCR in the cell,
thereby enhancing expression and/or formation of a therapeutic TCR modifying the cell for adoptive cell therapy,
or
a method for enhancing the sensitivity of an adoptive cell therapy TCR to an antigen, comprising modifying a cell expressing the adoptive cell therapy TCR by inactivating an endogenous TCRα chain allele and an endogenous TCRβ chain allele encoded by the cell, preferably by introducing at least one engineered nuclease to the cell, thereby enhancing the sensitivity of the adoptive cell therapy TCR to an antigen relative to an otherwise identical cell expressing the adoptive cell therapy TCR but not modified to inactivate an endogenous TCRα chain allele and an endogenous TCRβ chain allele encoded by the cell.
